SEQUENCE LISTING (35 sequences)

SEQ ID NO: 1 - DNA, 1259 bp, Papaver somniferum
atggagaaagcaaaacttatgaagctaggtaatggtatggaaataccaagtgttcaagaattggctaaactcacgcttgccgaaattccatctcgatacgtatgcaccgatgaaaaccttttgttgcctatgggtgcatctgtcataaatgatcatgaaaccattcctgtcatcgatatagaaaatttattatctccagaacccgtaatcggaaagttagaattggataggcttcattcttcttgcaaagaatggggtttttttcaggtagtgaaccatggagtcgacgcttcattggtggatagtgtaaaatcagaaattcaaggtttctttaacctttctatggatgagaaaactaaatatgaacaggaagatggagatgtagaaggatttggacaaggctttattgcatcagaggaccaaacacttgattgggcagatatatttatgatgttcactcttccactccatttaaggaagcctcacttattttcaaaactcccagtgcctctcagggagacaatcgaatcctactcatcagaaatgaaaaagttatccatggtcctctttaataagatggaaaaagctctacaagtacaagcagccgagattaagggtatgtcagaggtgtttaaagatgggacacaagcaatgaggatgaactattatcccccttgtcctcaaccaaatctcgccatcggtcttacgtcgcactcggattttggcggtttgacaatcctccttcaaatcaacgaagtggaaggattacagataaaaagagaggggacatggattttagtcaaacctctacctaatgcgttcgtagtgaatgttggagatattttggagataatgactaatggaatttaccatagcgtcgatcaccgggcagtagtaaactcaacaaatgagaggctctcaatcgcaacatttcatgaccctagtctagagtcagtaataggcccaatatcaagcttgattactccagagacacctgctttgtttaaaagtggatctacatatggggatcttgtggaggaatgtaaaacaaggaagctcgatggaaaatcatttcttgactccatgaggatgtgaaaactcaagaacacaataatacgaggtgattgcatgtcagattcaactatccttttgtcgttttttggtgctcgagtccttaattgttttcatcattgcttttgattctaattaataagacttttctcaagaactctcatgtaatgtacctttactttcagaaa

SEQ ID NO: 2 - DNA, 1259 bp, Papaver somniferum
ttggagaaagcaaaacttatgaagctaggtaatggtatggaaataccaagtgttcaagaattggctaaactcacgcttgccgaaattccatctcgatacgtatgcaccgatgaaaaccttttgttgcctatgggtgcatctgtcataaatgatcatgaaaccattcctgtcatcgatatagaaaatttattatctccagaacccgtaatcggaaagttagaattggataggcttcattctgcttgcaaagaatggggtttttttcaggtagtgaaccatggagtcgacgcttcattggtggatagtgtaaaatcagaaattcaaggtttctttaacctttctatggatgagaaaactaaatatgaacaggaagatggagatgtagaaggatttggacaaggctttattgcatcagaggaccaaacacttgattgggcagatatatttatgatgttcactcttccactccatttaaggaagcctcacttattttcaaaactcccagtgcctctcagggagacaatcgaatcctactcatcagaaatgaaaaagttatccatggtcctctttaataagatggaaaaagctctacaagtacaagcagccgagattaagggtatgtcagaggtgtttaaagatgggacacaagcaatgaggatgaactattatcccccttgtcctcaaccaaatctcgccatcggtcttacgtcgcactcggattttggcggtttgacaatcctccttcaaatcaacgaagtggaaggattacagataaaaagagaggggacatggattttagtcaaacctctacctaatgcgttcgtagtgaatgttggagatattttggagataatgactaatggaatttaccatagcgtcgatcaccgggcagtagtaaactcaacaaatgagaggctctcaatcgcaacatttcatgaccctagtctagagtcagtaataggcccaatatcaagcttgattactccagagacacctgctttgtttaaaagtggatctacatatggggatcttgtggaggaatgtaaaacaaggaagctcgatggaaaatcatttcttgactccatgaggatgtgaaaactcaagaacacaataatacgaggtgattgcatgtcagattcaactatccttttgtcgttttttggtgctcgagtccttaattgttttcatcattgcttttgattctaattaataagacttttctcaagaactctcatgtaatgtacctttactttcagaaa

SEQ ID NO: 3 - DNA, 1259 bp, Papaver somniferum
atggagaaagcaaaacttatgaagctaggtaatggtatggaaataccaagtgttcaagaattggctaaactcacgcttgccgaaattccatctcgatacgtatgcaccgatgaaaaccttttgttgcctatgggtgcttctgtcataaatgatcatgaaaccattcctgtcatcgatatagaaaatttattatctccagaacccataatcggaaagttagaattggataggcttcattctgcttgcaaagaatggggtttttttcaggtagtgaaccatggagtcgacgcttcattggtggatagtgtaaaatcagaaattcaaggtttctttaacctttctatggatgagaaaactaaatatgaacaggaagatggagatgtggaaggatttggacaaggctttattgcatcagaggaccaaacacttgattgggcagatatatttatgatgttcactcttccactccatttaaggaagcctcacttattttcaaaactcccagtgcctctcagggagacaatcgaatcctactcatcagaaatgaaaaacttatccatggttctctttaataagatggaaaaagctctacaagtacaagcagccgagattaagggtatgtcagaggtgtttaaagatgggacacaagcaatgaggatgaactattatcccccttgtcctcaaccaaatctcgccatcggtcttacgtcgcactcggattttggcggtttgacaatcctccttcaaatcaacgaagtggaaggattacagataaaaagagaggggacatggatttcagtcaaacctctacctaatgcgttcgtagtgaacgttggagatattttggagataatgactaatggaatttaccatagcgtcgatcaccgggcagtagtaaactcaacaaatgagaggctctcaatcgcaacatttcatgaccctagtctagagtcggtaataggcccaatatcaagcttgattactccagagacacctgctttgtttaaaagtggatctacatatggggatcttgtggaggaatgtaaaacaaggaagctcgatggaaaatcatttcttgactccatgaggatgtgaaaactcaagaacacaataatacgacgtgattgcatgtcagattcaactatccttttgtcgtttttttgtgctagagtccttaattgttttcatcattgcttttgattctaattaataagacttttctcaagaactcacatgtaatgtacctttactttcagaaa

SEQ ID NO: 4 - DNA, 1259 bp, Papaver somniferum
atggagaaagcaaaacttatgaagctaggtaatggtatggaaataccaagtgttcaagaattggctaaactcacgcttgccgaaattccatctcgatacgtatgcaccgatgaaaaccttttgttgcctatgggtgcatctgtcataaatgatcatgaaaccattcctgtcatcgatatagaaaatttattatctccagaacccgtaatcggaaagttagaattggataggcttcattcttcttgcaaagaatggggtttttttcaggtagtgaaccatggagtcgacgcttcattggtggatagtgtaaaatcagaaattcaaggtttctttaacctttctatggatgagaaaactaaatatgaacaggaagatggagatgtagaaggatttggacaaggctttattgcatcagaggaccaaacacttgattgggcagatatatttatgatgttcactcttccactccatttaaggaagcctcacttattttcaaaactcccagtgcctctcagggagacaatcgaatcctactcatcagaaatgaaaaagttatccatggtcctctttaataagatggaaaaagctctacaagtacaagcagccgagattaagggtatgtcagaggtgtttaaagatgggacacaagcaatgaggatgaactattatcccccttgtcctcaaccaaatctcgccatcggtcttacgtcgcactcggattttggcggtttgacaatcctccttcaaatcaacgaagtggaaggattacagataaaaagagaggggacatggattttagtcaaacctctacctaatgcgttcgtagtgaatgttggagatattttggagataatgactaatggaatttaccatagcgtcgatcaccgggcagtagtaaactcaacaaatgagaggctctcaatcgcaacatttcatgaccctagtctagagtcagtaataggcccaatatcaagcttgattactccagagacacctgctttgtttaaaagtggatctacatatggggatcttgtggaggaatgtaaaacaaggaagctcgatggaaaatcatttcttgactccatgaggatgtgaaaactcaagaacacaataatacgaggtgattgcatgtcagattcaactatccttttgtcgttttttggtgctcgagtccttaattgttttcatcattgcttttgattctaattaataagacttttctcaagaactcacatgtaatgtacctttactttcagaaa

SEQ ID NO: 5 - DNA, 2288 bp, Papaver somniferum
atggagaaagcaaaacttatgaagctaggtaatggtatggaaataccaagtgttcaagaattggctaaactcacgcttgccgaaattccatctcgatacgtatgcaccgatgaaaaccttttgttgcctatgggtgcatctgtcataaatgatcatgaaaccattcctgtcatcgatatagaaaatttattatctccagaacccgtaatcggaaagttagaattggataggcttcattcttcttgcaaagaatggggtttttttcaggtatatatgtcgtaaatacaagcattgtgattgcatccagtagttgtactttgttacactaaatattagctagtgaatcttcctttacatatatatgttgcaggtagtgaaccatggagtcgacgcttcattggtggatagtgtaaaatcagaaattcaaggtttctttaacctttctatggatgagaaaactaaatatgaacaggaagatggagatgtagaaggatttggacaaggctttattgcatcagaggaccaaacacttgattgggcagatatatttatgatgttcactcttccactccatttaaggaagcctcacttattttcaaaactcccagtgcctctcaggttattactaattcataatcggtttctatgcacttgactttttaatctcacattttcatagtcggtttctgtgcacctcactcagtttattcctattcttttttttttgtttggcccacacatatttaacatttagtccaccttgttagaatttcagactataaatcattctttacaattccttattagctgtaaccatgttgtatgtgaacatatttaatattttatgctgatatataattctgtatatgcatgctctagaattatatattctaccacttgtaggaagatacttccaattttatcttctcttattctttaatgatggaaaatttctttttaaacaagagctagaacggagtacacctcacctcatgccaggaaagacatatgtcattcaaccttccatcaagagttgaaaacaacctcacataggattatcataactagtatgtcattccaaatagtgtaagagacatttgtccttatgtgcccttaaacatacataattaacataaacaaactttaattcattgattagggttagtactaatcaagtttaaaggaatacaaattatgttttactgctgaaccaacataaaaaactgacacacctaagtctccctatttctgcaaaaaaaattatccctcttgatattattttctggttcccccattgaccaataaacatgcaaaattaacactctttaaaagagtatgcatgatagagcataatattttcctctctttgaattttctcagtgatgtgattggatacattaatttggttggtttattatagtttttcatatgattatctaatttacgtacagggagacaatcgaatcctactcatcagaaatgaaaaagttatccatggtcctctttaataagatggaaaaagctctacaagtacaagcagccgagattaagggtatgtcagaggtgtttaaagatgggacacaagcaatgaggatgaactattatcccccttgtcctcaaccaaatctcgccatcggtcttacgtcgcactcggattttggcggtttgacaatcctccttcaaatcaacgaagtggaaggattacagataaaaagagaggggacatggattttagtcaaacctctacctaatgcgttcgtagtgaatgttggagatattttggaggtatttatttaagctggggtaactactggtccttgtttcgaatactggttttgcctgtacccagaaaatttcttattgttttttcttttacagataatgactaatggaatttaccatagcgtcgatcaccgggcagtagtaaactcaacaaatgagaggctctcaatcgcaacatttcatgaccctagtctagagtcagtaataggcccaatatcaagcttgattactccagagacacctgctttgtttaaaagtggatctacatatggggatcttgtggaggaatgtaaaacaaggaagctcgatggaaaatcatttcttgactccatgaggatgtgaaaactcaagaacacaataatacgaggtgattgcatgtcagattcaactatccttttgtcgttttttggtgctcgagtccttaattgttttcatcattgcttttgattctaattaataagacttttctcaagaactctcatgtaatgtacctttactttcagaaa

SEQ ID NO: 6 - DNA, 2288 bp, Papaver somniferum
ttggagaaagcaaaacttatgaagctaggtaatggtatggaaataccaagtgttcaagaattggctaaactcacgcttgccgaaattccatctcgatacgtatgcaccgatgaaaaccttttgttgcctatgggtgcatctgtcataaatgatcatgaaaccattcctgtcatcgatatagaaaatttattatctccagaacccgtaatcggaaagttagaattggataggcttcattctgcttgcaaagaatggggtttttttcaggtatatatgtcgtaaatacaagcattgtgattgcacccagtagttgtactttgttacactaaatattagctagtgaatcttcctttacatatatatgttgcaggtagtgaaccatggagtcgacgcttcattggtggatagtgtaaaatcagaaattcaaggtttctttaacctttctatggatgagaaaactaaatatgaacaggaagatggagatgtagaaggatttggacaaggctttattgcatcagaggaccaaacacttgattgggcagatatatttatgatgttcactcttccactccatttaaggaagcctcacttattttcaaaactcccagtgcctctcaggttattactaattcataatcggtttctatgcacttgactttttaatctcacattttcatagtcggtttctgtgcacctcactcagtttattcctattcttttttttttgtttggcccacacatatttaacatttagtccaccttgttagaatttcagactataaatcattctttacaattccttattagctgtaaccatgttgtatgtgaacatatttaatattttatgctgatatataattctgtatatgcatgctctagaattatatattctaccacttgtaggaagatacttccaattttatcttctcttattctttaatgatggaaaatttctttttaaacaagagctagaacggagtacacctcacctcatgccaggaaagacatatgtcattcaaccttccatcaagagttgaaaacaacctcacataggattatcataactagtatgtcattccaaatagtgtaagagacatttgtccttatgtgcccttaaacatacataattaacataaacaaactttaattcattgattagggttagtactaatcaagtttaaaggaatacaaattatgttttactgctgaaccaacataaaaaactgacacacctaagtctccctatttctgcaaaaaaaattatccctcttgatattattttctggttcccccattgaccaataaacatgcaaaattaacactctttaaaagagtatgcatgatagagcataatattttcctctctttgaattttctcagtgatgtgattggatacattaatttggttggtttattatagtttttcatatgattatctaatttacgtacagggagacaatcgaatcctactcatcagaaatgaaaaagttatccatggtcctctttaataagatggaaaaagctctacaagtacaagcagccgagattaagggtatgtcagaggtgtttaaagatgggacacaagcaatgaggatgaactattatcccccttgtcctcaaccaaatctcgccatcggtcttacgtcgcactcggattttggcggtttgacaatcctccttcaaatcaacgaagtggaaggattacagataaaaagagaggggacatggattttagtcaaacctctacctaatgcgttcgtagtgaatgttggagatattttggaggtatttatttaagctggggtaactactggtccttgtttcgaatactggttttacctgtacccagaaaatttcttattgttttttcttttacagataatgactaatggaatttaccatagcgtcgatcaccgggcagtagtaaactcaacaaatgagaggctctcaatcgcaacatttcatgaccctagtctagagtcagtaataggcccaatatcaagcttgattactccagagacacctgctttgtttaaaagtggatctacatatggggatcttgtggaggaatgtaaaacaaggaagctcgatggaaaatcatttcttgactccatgaggatgtgaaaactcaagaacacaataatacgaggtgattgcatgtcagattcaactatccttttgtcgttttttggtgctcgagtccttaattgttttcatcattgcttttgattctaattaataagacttttctcaagaactctcatgtaatgtacctttactttcagaaa

SEQ ID NO: 7 - DNA, 2286 bp, Papaver somniferum
atggagaaagcaaaacttatgaagctaggtaatggtatggaaataccaagtgttcaagaattggctaaactcacgcttgccgaaattccatctcgatacgtatgcaccgatgaaaaccttttgttgcctatgggtgcttctgtcataaatgatcatgaaaccattcctgtcatcgatatagaaaatttattatctccagaacccataatcggaaagttagaattggataggcttcattctgcttgcaaagaatggggtttttttcaggtatatatgtcgtaaatacaagcattgtgattgcatccagtagttgtactttgttacactaaatattatctagtgaatcttcctttacatatatatgttgcaggtagtgaaccatggagtcgacgcttcattggtggatagtgtaaaatcagaaattcaaggtttctttaacctttctatggatgagaaaactaaatatgaacaggaagatggagatgtggaaggatttggacaaggctttattgcatcagaggaccaaacacttgattgggcagatatatttatgatgttcactcttccactccatttaaggaagcctcacttattttcaaaactcccagtgcctctcaggttattattcattcataatcgatttctatgcacttgactttttaatctcacattttcatagtcggtttatgtgcacctcactcagtttattcttattctttttttttgtttggcccacacatatttaacatttagtccaccttgttagaatttcagactataaatcattctttacaattccttattagctgtaaccatgttgtatgtgaacatatttaatattttatgcttatatataattctgtatatgcatgctctagaattatatattctaccacttgtaggaagatacttccaattttatcttctcttattcattaatgatggaaaattcctttttaaacaagagctagaacggagtacaccttatctcatgctaggaaagacatatatcattcaaccttccatcaagagttgaaaacaacctcacataggattatcataaatagtatgtcattccaaatagtgtaagagatatttgtccttatgtacccttaaacatacataattaacataaacaaaccttaattcattgattaggattaatactaatcaagtttaaagtaatataaattatgttttactaccgaaccaacataaaaaactgacacacctaagtctccctatttctataaaaaaattatcccttttgatattattttctggttcccccattgaccaataaacatgcaaaactaacactctgtaaaagagtatgcatgatagatcataatatttttctctctttgaattttctcagtgatgtgattggatacattaatttggttggtttattatagtttttcatatgattatctaatttacgtacagggagacaatcgaatcctactcatcagaaatgaaaaacttatccatggttctctttaataagatggaaaaagctctacaagtacaagcagccgagattaagggtatgtcagaggtgtttaaagatgggacacaagcaatgaggatgaactattatcccccttgtcctcaaccaaatctcgccatcggtcttacgtcgcactcggattttggcggtttgacaatcctccttcaaatcaacgaagtggaaggattacagataaaaagagaggggacatggatttcagtcaaacctctacctaatgcgttcgtagtgaacgttggagatattttggaggtatttatttaagcttgggtaactactggtccttgtttcgaatacctgtttttcctgtacccagaaaatttcttattgttttttcttttatagataatgactaatggaatttaccatagcgtcgatcaccgggcagtagtaaactcaacaaatgagaggctctcaatcgcaacatttcatgaccctagtctagagtcggtaataggcccaatatcaagcttgattactccagagacacctgctttgtttaaaagtggatctacatatggggatcttgtggaggaatgtaaaacaaggaagctcgatggaaaatcatttcttgactccatgaggatgtgaaaactcaagaacacaataatacgacgtgattgcatgtcagattcaactatccttttgtcgtttttttgtgctagagtccttaattgttttcatcattgcttttgattctaattaataagacttttctcaagaactcacatgtaatgtacctttactttcagaaa

SEQ ID NO: 8 - DNA, 2288 bp, Papaver somniferum
atggagaaagcaaaacttatgaagctaggtaatggtatggaaataccaagtgttcaagaattggctaaactcacgcttgccgaaattccatctcgatacgtatgcaccgatgaaaaccttttgttgcctatgggtgcatctgtcataaatgatcatgaaaccattcctgtcatcgatatagaaaatttattatctccagaacccgtaatcggaaagttagaattggataggcttcattcttcttgcaaagaatggggtttttttcaggtatatatgtcgtaaatacaagcattgtgattgcatccagtagttgtactttgttacactaaatattagctagtgaatcttcctttacatatatatgttgcaggtagtgaaccatggagtcgacgcttcattggtggatagtgtaaaatcagaaattcaaggtttctttaacctttctatggatgagaaaactaaatatgaacaggaagatggagatgtagaaggatttggacaaggctttattgcatcagaggaccaaacacttgattgggcagatatatttatgatgttcactcttccactccatttaaggaagcctcacttattttcaaaactcccagtgcctctcaggttattactaattcataatcggtttctatgcacttgactttttaatctcacattttcatagtcggtttctgtgcacctcactcagtttattcctattcttttttttttgtttggcccacacatatttaacatttagtccaccttgttagaatttcagactataaatcattctttacaattccttattagctgtaaccatgttgtatgtgaacatatttaatattttatgctgatatataattctgtatatgcatgctctagaattatatattctaccacttgtaggaagatacttccaattttatcttctcttattctttaatgatggaaaatttctttttaaacaagagctagaacggagtacacctcacctcatgccaggaaagacatatgtcattcaaccttccatcaagagttgaaaacaacctcacataggattatcataactagtatgtcattccaaatagtgtaagagacatttgtccttatgtgcccttaaacatacataattaacataaacaaactttaattcattgattagggttagtactaatcaagtttaaaggaatacaaattatgttttactgctgaaccaacataaaaaactgacacacctaagtctccctatttctgcaaaaaaaattatccctcttgatattattttctggttcccccattgaccaataaacatgcaaaattaacactctttaaaagagtatgcatgatagagcataatattttcctctctttgaattttctcagtgatgtgattggatacattaatttggttggtttattatagtttttcatatgattatctaatttacgtacagggagacaatcgaatcctactcatcagaaatgaaaaagttatccatggtcctctttaataagatggaaaaagctctacaagtacaagcagccgagattaagggtatgtcagaggtgtttaaagatgggacacaagcaatgaggatgaactattatcccccttgtcctcaaccaaatctcgccatcggtcttacgtcgcactcggattttggcggtttgacaatcctccttcaaatcaacgaagtggaaggattacagataaaaagagaggggacatggattttagtcaaacctctacctaatgcgttcgtagtgaatgttggagatattttggaggtatttatttaagctggggtaactactggtccttgtttcgaatactggttttgcctgtacccagaaaatttcttattgttttttcttttacagataatgactaatggaatttaccatagcgtcgatcaccgggcagtagtaaactcaacaaatgagaggctctcaatcgcaacatttcatgaccctagtctagagtcagtaataggcccaatatcaagcttgattactccagagacacctgctttgtttaaaagtggatctacatatggggatcttgtggaggaatgtaaaacaaggaagctcgatggaaaatcatttcttgactccatgaggatgtgaaaactcaagaacacaataatacgaggtgattgcatgtcagattcaactatccttttgtcgttttttggtgctcgagtccttaattgttttcatcattgcttttgattctaattaataagacttttctcaagaactcacatgtaatgtacctttactttcagaaa

SEQ ID NO: 9 - protein, 364 aa, Papaver somniferum
MEKAKLMKLGNGMEIPSVQELAKLTLAEIPSRYVCTDENLLLPMGASVINDHETIPVIDIENLLSPEPVIGKLELDRLHSSCKEWGFFQVVNHGVDASLVDSVKSEIQGFFNLSMDEKTKYEQEDGDVEGFGQGFIASEDQTLDWADIFMMFTLPLHLRKPHLFSKLPVPLRETIESYSSEMKKLSMVLFNKMEKALQVQAAEIKGMSEVFKDGTQAMRMNYYPPCPQPNLAIGLTSHSDFGGLTILLQINEVEGLQIKREGTWILVKPLPNAFVVNVGDILEIMTNGIYHSVDHRAVVNSTNERLSIATFHDPSLESVIGPISSLITPETPALFKSGSTYGDLVEECKTRKLDGKSFLDSMRM

SEQ ID NO: 10 - protein, 364 aa, Papaver somniferum
LEKAKLMKLGNGMEIPSVQELAKLTLAEIPSRYVCTDENLLLPMGASVINDHETIPVIDIENLLSPEPVIGKLELDRLHSACKEWGFFQVVNHGVDASLVDSVKSEIQGFFNLSMDEKTKYEQEDGDVEGFGQGFIASEDQTLDWADIFMMFTLPLHLRKPHLFSKLPVPLRETIESYSSEMKKLSMVLFNKMEKALQVQAAEIKGMSEVFKDGTQAMRMNYYPPCPQPNLAIGLTSHSDFGGLTILLQINEVEGLQIKREGTWILVKPLPNAFVVNVGDILEIMTNGIYHSVDHRAVVNSTNERLSIATFHDPSLESVIGPISSLITPETPALFKSGSTYGDLVEECKTRKLDGKSFLDSMRM

SEQ ID NO: 11 - protein, 364 aa, Papaver somniferum
MEKAKLMKLGNGMEIPSVQELAKLTLAEIPSRYVCTDENLLLPMGASVINDHETIPVIDIENLLSPEPIIGKLELDRLHSACKEWGFFQVVNHGVDASLVDSVKSEIQGFFNLSMDEKTKYEQEDGDVEGFGQGFIASEDQTLDWADIFMMFTLPLHLRKPHLFSKLPVPLRETIESYSSEMKNLSMVLFNKMEKALQVQAAEIKGMSEVFKDGTQAMRMNYYPPCPQPNLAIGLTSHSDFGGLTILLQINEVEGLQIKREGTWISVKPLPNAFVVNVGDILEIMTNGIYHSVDHRAVVNSTNERLSIATFHDPSLESVIGPISSLITPETPALFKSGSTYGDLVEECKTRKLDGKSFLDSMRM

SEQ ID NO: 12 - DNA, 1083 bp, Papaver somniferum
atggagacaccaatacttatcaagctaggcaatggtttgtcaataccaagtgttcaggaattggctaaactcacgcttgcagaaattccatctcgatacacatgcaccggtgaaagcccgttgaataatattggtgcgtctgtaacagatgatgaaacagttcctgtcatcgatttgcaaaatttactatctccagaacccgtagttggaaagttagaattggataagcttcattctgcttgcaaagaatggggtttctttcagctggttaaccatggagtcgacgctttactgatggacaatataaaatcagaaattaaaggtttctttaaccttccaatgaatgagaaaactaaatacggacagcaagatggagattttgaaggatttggacaaccctatattgaatcggaggaccaaagacttgattggactgaagtgtttagcatgttaagtcttcctctccatttaaggaagcctcatttgtttccagaactccctctgcctttcagggagacactggaatcctacctatcaaaaatgaaaaaactatcaacggttgtctttgagatgttggaaaaatctctacaattagttgagattaaaggtatgacagacttatttgaagatgggttgcaaacaatgaggatgaactattatcctccttgtcctcgaccagagcttgtacttggtcttacgtcacactcggattttagcggtttgacaattctccttcaacttaatgaagttgaaggattacaaataagaaaagaagagaggtggatttcaatcaaacctctacctgatgcgttcatagtgaatgttggagacattttggagataatgactaatgggatttaccgtagcgtcgagcaccgggcagtagtaaactcaacaaaggagaggctctcaatcgcgacatttcatgactctaaactagagtcagaaataggcccaatttctagcttggtcacaccagagacacctgctttgttcaaaagaggtaggtatgaggatattttgaaggaaaatctttcaaggaagcttgatggaaaatcatttctcgactacatgaggatgtga

SEQ ID NO: 13 - DNA, 1663 bp, Papaver somniferum
atctgacaagaaagttcatcaaatatagagttcatggagacaccaatacttatcaagctaggcaatggtttgtcaataccaagtgttcaggaattggctaaactcacgcttgcagaaattccatctcgatacacatgcaccggtgaaagcccgttgaataatattggtgcgtctgtaacagatgatgaaacagttcctgtcatcgatttgcaaaatttactatctccagaacccgtagttggaaagttagaattggataagcttcattctgcttgcaaagaatggggtttctttcaggtatacaaattttgattatacgtagttgattgacgactatttagtgttttactagttcatgctaatctgctttatgtttcagctggttaaccatggagtcgacgctttactgatggacaatataaaatcagaaattaaaggtttctttaaccttccaatgaatgagaaaactaaatacggacagcaagatggagattttgaaggatttggacaaccctatattgaatcggaggaccaaagacttgattggactgaagtgtttagcatgttaagtcttcctctccatttaaggaagcctcatttgtttccagaactccctctgcctttcaggttattgtttctctccgtttgctttttctcaatgccgcacgtgatattttttttttttaatgttattttggttggttcagaatagtttcatcaatatcgtctaggttgctaggataaaccatggaaaatcctgtctaggttgcctaaaattcacattcggaggcagtatcaagcaatccgggtttgtgaattgcatggtttccaaaatatatcgaatccaaacatcatgcatccctaaaatctgtcagatggaaaatatactgaagttgcatttgatccctaatttacatcctttatgcagggagacactggaatcctacctatcaaaaatgaaaaaactatcaacggttgtctttgagatgttggaaaaatctctacaattagttgagattaaaggtatgacagacttatttgaagatgggttgcaaacaatgaggatgaactattatcctccttgtcctcgaccagagcttgtacttggtcttacgtcacactcggattttagcggtttgacaattctccttcaacttaatgaagttgaaggattacaaataagaaaagaagagaggtggatttcaatcaaacctctacctgatgcgttcatagtgaatgttggagacattttggaggtatttaggctaagggcatatatggcctgcctactggtccttgttgtgagtacctgttttgactgtaccccgaaaagttgcttagttacttaatgtttcccttttacagataatgactaatgggatttaccgtagcgtcgagcaccgggcagtagtaaactcaacaaaggagaggctctcaatcgcgacatttcatgactctaaactagagtcagaaataggcccaatttctagcttggtcacaccagagacacctgctttgttcaaaagaggtaggtatgaggatattttgaaggaaaatctttcaaggaagcttgatggaaaatcatttctcgactacatgaggatgtgagaaagtgtgaacatatattatactccacattgtgtttaatatatgatgaaataag

SEQ ID NO: 14 - DNA, 769 bp, Artificial Sequence
  CaMV 35S dual enhancer promoter sequence
caggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgactctagaggatc

SEQ ID NO: 15 - DNA, 33 bp, Artificial Sequence
  F1 PCR primer (Example 2)
atctgacaagaaagttcatcaaatatagagttc

SEQ ID NO: 16 - DNA, 27 bp, Artificial Sequence
  R4 PCR primer (Example 2)
gtaggattcsaktgtctccctgaragg

SEQ ID NO: 17 - DNA, 29 bp, Artificial Sequence
  R5 PCR primer (Example 2)
aacccatcttcaaataagtctgtcatacc

SEQ ID NO: 18 - DNA, 28 bp, Artificial Sequence
  R7 PCR primer (Example 2)
tytcamatcctcatgkagtcragaaatg

SEQ ID NO: 19 - DNA, 33 bp, Artificial Sequence
  R8 PCR primer (Example 2)
cttatttcatcatatattaaacacaatgtggag

SEQ ID NO: 20 - DNA, 29 bp, Artificial Sequence
  F2 PCR primer (Example 2)
atggagamascaawacttatsaagctagg

SEQ ID NO: 21 - DNA, 29 bp, Artificial Sequence
  R6 PCR primer (Example 2)
gtcccatctataaacacctctgacatacc

SEQ ID NO: 22 - DNA, 30 bp, Artificial Sequence
  R9 PCR primer (Example 2)
tttctgaaagtaaaggtacattacatgtgg

SEQ ID NO: 23 - DNA, 26 bp, Artificial Sequence
  F3 PCR primer (Example 2)
gracagsaagatggagatktkgaagg

SEQ ID NO: 24 - DNA, 31 bp, Artificial Sequence
  F10 PCR primer (Example 2)
atggagagtaatggtgtacctatgatcactc

SEQ ID NO: 25 - DNA, 30 bp, Artificial Sequence
  R11 PCR primer (Example 2)
tcaatccttctcatcccagaactcttcttc

SEQ ID NO: 26 - DNA, 29 bp, Artificial Sequence
  T6ODM PCR amplification primer-1 (Example 3)
atggagaaagcaaaacttatgaagctagg

SEQ ID NO: 27 - DNA, 30 bp, Artificial Sequence; identical to SEQ ID NO: 22
  T6ODM PCR amplification primer-2 (Example 3)

SEQ ID NO: 28 - DNA, 33 bp, Artificial Sequence; identical to SEQ ID NO: 15
  CODM PCR amplification primer-1 (Example 3)

SEQ ID NO: 29 - DNA, 33 bp, Artificial Sequence; identical to SEQ ID NO: 19
  CODM PCR amplification primer-2 (Example 3)

SEQ ID NO: 30 - DNA, 30 bp, Artificial Sequence
  CaMV 35S promoter PCR primer-1 (Example 4)
caaacgcgtcaggtcaacatggtggagcac

SEQ ID NO: 31 - DNA, 34 bp, Artificial Sequence
  CaMV 35S promoter PCR primer-2 (Example 4)
caaagatctgatcctctagagtcgaggtcctctc

SEQ ID NO: 32 - DNA, 33 bp, Artificial Sequence
  pat gene PCR amplification primer-1 (Example 4)
gatctcgagctttcgatgagcccagaacgacgc

SEQ ID NO: 33 - DNA, 41 bp, Artificial Sequence
  pat gene PCR amplification primer-2 (Example 4)
aaactcgagcttgtcgatcgacatcagatttcggtgacggg

SEQ ID NO: 34 - DNA, 33 bp, Artificial Sequence
  als gene PCR amplification primer-1 (Example 4)
gttctcgagctttcgatggcagccaccaccacc

SEQ ID NO: 35 - DNA, 47 bp, Artificial Sequence
  als gene PCR amplification primer-2 (Example 4)
ttactcgagcttgtcgatcgacatcaataactccttcttccatcacc

(19) United States
(12) Patent Application Publication
(10) Pub. No.: US 2014/0013465 A1
(43) Pub. Date: Jan. 9, 2014

(54) PAPAVER BRACTEATUM WITH MODIFIED ALKALOID CONTENT

(71) Applicant: TPI ENTERPRISES LTD., Launceston (AU)

(72) Inventors: Justin Taylor Coombs, Wattle Park (AU); Jarrod David Ritchie, Launceston (AU); Mark Alfred Tester, Toorak Gardens (AU); Damien Lightfoot, Netherby (AU); Deepa Jha, Magill (AU)

(73) Assignee: TPI ENTERPRISES LTD., LAUNCESTON (AU)

(21) Appl. No.: 13/834,979

(22) Filed: Mar. 15, 2013

Related U.S. Application Data
(63) Continuation-in-part of application No. PCT/AU2011/001400, filed on Oct. 31, 2011.
Application No. PCT/AU2011/001400 is a continuation of application No. PCT/AU2012/000457, filed on Apr. 30, 2012.
(60) Provisional application No. 61/640,160, filed on Apr. 30, 2012.

(30) Foreign Application Priority Data
Nov. 1, 2010 (AU) 2010904872

(51) Int. Cl. C12N 15/82; C07D 221/28; C12N 9/02; A01H 5/00; C07D 489/02

(52) U.S. Cl. CPC C12N 15/8243; C07D 221/28; C12N 9/0071
USPC 800/278; 435/410; 435/122; 546/44

(57) ABSTRACT

The present invention relates to genetically modified plants of the species Papaver bracteatum wherein the type or amount of one or more alkaloids produced by the plants has been modified. Specifically, the genetically modified plants have an increased expression of one or more of thebaine 6-O-demethylase, codeine O-demethylase and/or codeinone reductase relative to wild type P. bracteatum such that the genetically modified poppy plants produce an increased quantity of an alkaloid selected from codeine, oripavine and/or morphine relative to a wild type P. bracteatum. Also provided are progeny plants having the genetically modified poppy plants described above as a parent; mutant or derivative plants of the aforementioned plants; reproductive material derived from, straw produced from, straw concentrate produced from, latex derived from, or one or more isolated cells derived from, the aforementioned plants. Methods for producing an alkaloid from the aforementioned plants are also provided, together with nucleic acid and amino acid sequence variants of the 6-O-demethylase and codeine O-demethylase genes.

### CROSS REFERENCE TO RELATED APPLICATIONS

This is a continuation-in-part of International Application No. PCT/AU2011/001400, filed Oct. 31, 2011, which claims the benefit of AU2010904872, filed Nov. 1, 2010; and is also a continuation of International Application No. PCT/AU2012/000457, filed Apr. 30, 2012; and further claims the benefit of U.S. Provisional Application No. 61/640,160, filed Apr. 30, 2012. All of these are incorporated by reference herein in their entirety.

### FIELD OF THE INVENTION

The present invention relates to genetically modified plants of the species Papaver bracteatum wherein the type or amount of one or more alkaloids produced by the plant has been modified.

### BACKGROUND OF THE INVENTION

Opium poppies (Papaver somniferum) are commercially cultivated in a number of countries under regulatory control. The latex obtained by the incision of unripe capsules is known as opium and is the source of several pharmacologically important alkaloids. Morphine, codeine, thebaine, narcotine and papaverine are the most important alkaloids produced by the plant, and are exploited by the pharmaceutical industry as analgesics, antitussives and antispasmodics.

On a commercial scale, however, poppy straw and straw concentrate (rather than latex) are the most commonly used raw materials for the production of morphine and other poppy-derived alkaloids. For example, more than 50% of the world's morphine is now manufactured from poppy straw or poppy straw concentrate.

Australia produces a significant portion (approximately 30% in 2006) of the world's poppy straw concentrates for morphine production. Furthermore, the Australian State of Tasmania is the world's largest producer of opium alkaloids for the pharmaceutical market. The area sown to poppies in Tasmania was close to 34,000 ha in 2011.

At present, valuable alkaloids such as morphine are produced in the annual poppy species, Papaver somniferum.

However, the present inventors have recognized that perennial plant species provide a number of agronomic benefits over annual species, such as increased hardiness, increased water and/or nutrient use efficiency, elimination of the necessity to replant every season, reduction in risks associated with crop establishment and a substantial reduction in the necessity for crop rotation. It has been recognized one or more of the above-listed benefits would have the potential to increasing overall alkaloid production and/or efficiency.

Papaver bracteatum (herein referred to as P. bracteatum) is one perennial species of poppy which produces significant quantities of the alkaloid thebaine. Thebaine itself is not used therapeutically, however, this alkaloid is used as a feedstock for the production of other alkaloids, including the production of oxycodone.

However, it is recognized in the art that P. bracteatum does not produce significant quantities of other commercially valuable alkaloids such as codeine, oripavine and/or morphine.

Furthermore, although mutagenesis has been used to alter the type of alkaloids produced by the annual poppy species, Papaver somniferum (e.g. see WO 98/02033), the inventors are not aware of any reports describing an alteration of the alkaloid profile of P. bracteatum.

A further significant problem affecting the cultivation of P. bracteatum in at least some poppy growing regions (including Tasmania, Australia) is that many varieties do not produce seed capsules and significant quantities of alkaloid in the first growing season after seed germination.

Accordingly, perennial poppy plants (such as of the species P. bracteatum) that produce extractable quantities of one or more of the alkaloids codeine, oripavine and/or morphine would be highly desirable. Furthermore, the ability of such cultivars to produce alkaloids such as morphine and/or codeine in the first growing season after germination in at least temperate poppy-growing regions (such as Tasmania, Australia) would also be desirable.

Reference to any prior art in this specification is not, and should not be taken as, an acknowledgment or any form of suggestion that this prior art forms part of the common general knowledge in any country.

### SUMMARY OF THE INVENTION

In a first aspect, the present invention provides a genetically modified poppy plant of the species Papaver bracteatum, or a hybrid thereof, wherein the expression of one or more of thebaine 6-O-demethylase, codeine O-demethylase and/or codeinone reductase is increased relative to wild type P. bracteatum and wherein said genetically modified poppy plant produces an increased quantity of an alkaloid selected from codeine, oripavine and/or morphine relative to a wild type P. bracteatum.

In one embodiment, at least the expression of thebaine 6-O-demethylase is increased relative to wild type P. bracteatum.

In some embodiments, the poppy plant comprises one or more transgenes which encode thebaine 6-O-demethylase, codeine O-demethylase and/or codeinone reductase. In some embodiments, the one or more transgenes comprise the nucleotide sequence set forth in any one or more of SEQ ID NOs: 1 to 8, 12 and 13.

In some embodiments, the thebaine 6-O-demethylase comprises the amino acid sequence set forth in one of SEQ ID NOs: 9 to 11.

In some embodiments, the poppy plant produces seed capsules and an alkaloid selected from codeine, oripavine and/or morphine in the first growing season after germination in a temperate poppy growing region.

In some embodiments, the poppy plant produces seed capsules and an alkaloid selected from codeine, oripavine and/or morphine in the first growing season after germination at a latitude of between 40° and 44°.

In some embodiments, the poppy plant produces seed capsules and an alkaloid selected from codeine, oripavine and/or morphine in the first growing season after germination in Tasmania, Australia.

In some embodiments, the poppy plant comprises an indehiscent seed capsule or a dehiscent seed capsule.

In a second aspect, the present invention provides a progeny plant having a poppy plant of a first aspect of the invention as a parent, wherein the progeny plant comprises increased expression of one or more of thebaine 6-O-demethylase, codeine O-demethylase and/or codeinone reductase relative to wild type P. bracteatum and wherein said progeny plant produces an increased quantity of an alkaloid selected from codeine, oripavine and/or morphine relative to a wild type P. bracteatum.

In a third aspect, the present invention provides a mutant or derivative plant of the plant of a first or second aspect of the invention wherein the mutant or derivative comprises increased expression of one or more of thebaine 6-O-demethylase, codeine O-demethylase and/or codeinone reductase relative to wild type P. bracteatum and wherein said mutant or derivative produces an increased quantity of an alkaloid selected from codeine, oripavine and/or morphine relative to a wild type P. bracteatum.

In a fourth aspect, the present invention provides reproductive material derived from the plant of any one of the first, second or third aspects of the invention. In some embodiments, the reproductive material comprises a seed.

In a fifth aspect, the present invention provides straw produced from the plant of any one of the first, second or third aspects of the invention.

In a sixth aspect, the present invention provides a straw concentrate produced from the plant of any one of the first, second or third aspects of the invention.

In a seventh aspect, the present invention provides latex derived from the plant of any one of the first, second or third aspects of the invention.

In an eighth aspect, the present invention provides a stand of stably reproducing poppy plants of any one of the first, second or third aspects of the invention.

In a ninth aspect, the present invention provides an isolated cell derived from the plant of any one of the first, second or third aspects of the invention.

In a tenth aspect, the present invention provides an in-vitro culture comprising one or more of the cells of ninth aspect of the invention. In one embodiment, said culture produces an alkaloid selected from codeine, oripavine and/or morphine.

In an eleventh aspect, the present invention provides a method of producing an alkaloid selected from codeine, oripavine and/or morphine, the method comprising growing a plant of any one of the first, second or third aspects of the invention such that the plant produces an alkaloid selected from codeine, oripavine and/or morphine; and extracting the codeine, oripavine and/or morphine from the poppy plant or a part thereof.

In a twelfth aspect, the present invention provides an alkaloid selected from codeine, oripavine and/or morphine produced according to the method of the eleventh aspect of the invention.

In a thirteenth aspect, the present invention provides an expression construct comprising one or more transgenes which encode thebaine 6-O-demethylase, codeine O-demethylase and/or codeinone reductase, wherein said one or more transgenes are operably connected to a transcriptional control sequence which is active in P. bracteatum.

In some embodiments, the one or more transgenes comprise the nucleotide sequence set forth in any one or more of SEQ ID NOs: 1 to 8, 12 and 13.

In some embodiments of the thirteenth aspect of the invention, the thebaine 6-O-demethylase comprises the amino acid sequence set forth in one of SEQ ID NOs: 9 to 11.

In a fourteenth aspect, the present invention provides an isolated nucleic acid molecule comprising the nucleotide sequence set forth in any one of SEQ ID NOs: 1 to 8, 12 and 13.

In a fifteenth aspect, the present invention provides an isolated nucleic acid molecule selected from the group consisting of:
(i) a nucleic acid molecule comprising a nucleotide sequence which is at least 50% identical to the nucleotide sequence set forth in any one of SEQ ID NOs: 1 to 8, 12 and 13; and
(ii) a nucleic acid molecule which hybridizes to a nucleic acid molecule comprising the nucleotide sequence set forth in any one of SEQ ID NOs: 1 to 8, 12 and 13 under stringent conditions.

In a sixteenth aspect, the present invention provides an isolated polypeptide comprising the amino acid sequence set forth in one of SEQ ID NOs: 9 to 11.

In a seventeenth aspect, the present invention provides an isolated polypeptide comprising an amino acid sequence which is at least 50% identical to the amino acid sequence set forth in any one of SEQ ID NOs: 9 to 11.

### BRIEF DESCRIPTION OF THE FIGURES

FIG. 1 is a schematic diagram showing the biosynthesis of morphine from thebaine in Papaver somniferum. T6ODM—thebaine 6-O-demethylase; CODM—codeine O-demethylase; COR—codeinone reductase.

FIG. 2 is a photograph of an electrophoresis gel showing the products of PCR amplification of DNA isolated from P. somniferum and P. bracteatum using primers to the T6ODM and CODM genes. The primer combinations corresponding to each lane in the gel are provided in Table 2. P. somniferum genomic DNA as a template (top panel); P. bracteatum genomic DNA as a template (bottom panel).

FIG. 3 is a schematic showing the location of the primers in the CODM and T6ODM genes used to PCR amplify the products shown in FIG. 2.

FIG. 4 is a nucleotide sequence alignment of T6ODM variants (clones 21, 22, 23 and 11) with a reference cDNA sequence (GQ500139) for T6ODM (referred to as “cDNA” in the alignment). Coding sequences are boxed in black and intronic sequences are boxed in grey. Nucleotide sequence differences are boxed in white.

FIG. 5 is an amino acid sequence alignment of T6ODM variants encoded by the nucleotide sequences of FIG. 4. The amino acid sequence of variant clones 21, 22 and 23 is shown compared to the amino acid sequence encoded by the reference cDNA sequence (GQ500139) for T6ODM (referred to as “cDNA” in the alignment).

FIG. 6 is a nucleotide sequence alignment of a CODM variant (clone 21) with a reference cDNA sequence (GQ500141) for CODM (referred to as “cDNA” in the alignment). Coding sequences are boxed in black and intronic sequences are boxed in grey. The nucleotide sequence difference is boxed in white.

FIG. 7 is a schematic showing a vector map of the T6ODM construct generated in Example 4.

FIG. 8 is a schematic showing a vector map of the bar (Basta) construct generated in Example 4.

FIG. 9 is a schematic showing a vector map of the CODM construct generated in Example 4.

FIG. 10 is a schematic showing a vector map of the COR1.1 construct generated in Example 4.

FIG. 11 provides photos evidencing callus induction and plant regeneration of P. bracteatum var PB-1. (A) Callus from seed after 8 weeks: (B) shoot regeneration after 20 weeks; (C) plantlets with roots after 24 weeks; (D, E and F) new callus growth under selection after agrobacterium mediated transformation with 35S::Basta, 35S::CODM, and 35S::T6ODM, respectively.

### DESCRIPTION OF THE INVENTION

Nucleotide and amino acid sequences are referred to herein by a sequence identifier number (SEQ ID NO:). A summary of the sequence identifiers is provided in Table 1. The Sequence Listing is submitted as an ASCII text file in the form of the file named Sequence_Listing.txt, which was created on Mar. 13, 2013, and is 39,975 bytes, which is incorporated by reference herein.

TABLE 1
Summary of Sequence Identifiers

| Sequence | | Sequence |
|---|---|---|
| Identifier | Sequence | Listing Number |
| SEQ ID NO: 1 | T6ODM variant (clone 21) - cDNA nucleotide sequence | 400 <1> |
| SEQ ID NO: 2 | T6ODM variant (clone 22) - cDNA nucleotide sequence | 400 <2> |
| SEQ ID NO: 3 | T6ODM variant (clone 23) - cDNA nucleotide sequence | 400 <3> |
| SEQ ID NO: 4 | T6ODM variant (clone 11) - cDNA nucleotide sequence | 400 <4> |
| SEQ ID NO: 5 | T6ODM variant (clone 21) - genomic DNA sequence | 400 <5> |
| SEQ ID NO: 6 | T6ODM variant (clone 22) - genomic DNA sequence | 400 <6> |
| SEQ ID NO: 7 | T6ODM variant (clone 23) - genomic DNA sequence | 400 <7> |
| SEQ ID NO: 8 | T6ODM variant (clone 11) - genomic DNA sequence | 400 <8> |
| SEQ ID NO: 9 | T6ODM variant (clone 21) - amino acid sequence | 400 <9> |
| SEQ ID NO: 10 | T6ODM variant (clone 22) - amino acid sequence | 400 <10> |
| SEQ ID NO: 11 | T6ODM variant (clone 23) - amino acid sequence | 400 <11> |
| SEQ ID NO: 12 | CODM variant (clone 21) - cDNA nucleotide sequence | 400 <12> |
| SEQ ID NO: 13 | CODM variant (clone 21) - genomic DNA sequence | 400 <13> |
| SEQ ID NO: 14 | CaMV 35S dual enhancer nucleotide sequence | 400 <14> |
| SEQ ID NO: 15 | F1 PCR primer (Example 2) | 400 <15> |
| SEQ ID NO: 16 | R4 PCR primer (Example 2) | 400 <16> |
| SEQ ID NO: 17 | R5 PCR primer (Example 2) | 400 <17> |
| SEQ ID NO: 18 | R7 PCR primer (Example 2) | 400 <18> |
| SEQ ID NO: 19 | R8 PCR primer (Example 2) | 400 <19> |
| SEQ ID NO: 20 | F2 PCR primer (Example 2) | 400 <20> |
| SEQ ID NO: 21 | R6 PCR primer (Example 2) | 400 <21> |
| SEQ ID NO: 22 | R9 PCR primer (Example 2) | 400 <22> |
| SEQ ID NO: 23 | F3 PCR primer (Example 2) | 400 <23> |
| SEQ ID NO: 24 | F10 PCR primer (Example 2) | 400 <24> |
| SEQ ID NO: 25 | R11 PCR primer (Example 2) | 400 <25> |
| SEQ ID NO: 26 | T6ODM PCR amplification primer-1 (Example 3) | 400 <26> |
| SEQ ID NO: 27 | T6ODM PCR amplification primer-2 (Example 3) | 400 <27> |
| SEQ ID NO: 28 | CODM PCR amplification primer-1 (Example 3) | 400 <28> |
| SEQ ID NO: 29 | CODM PCR amplification primer-2 (Example 3) | 400 <29> |
| SEQ ID NO: 30 | CMV 35S promoter PCR primer-1 (Example 4) | 400 <30> |
| SEQ ID NO: 31 | CMV 35S promoter PCR primer-2 (Example 4) | 400 <31> |
| SEQ ID NO: 32 | pat gene PCR amplification primer-1 (Example 4) | 400 <32> |
| SEQ ID NO: 33 | pat gene PCR amplification primer-2 (Example 4) | 400 <33> |
| SEQ ID NO: 34 | als gene PCR amplification primer-1 (Example 4) | 400 <34> |
| SEQ ID NO: 35 | als gene PCR amplification primer-2 (Example 4) | 400 <35> |

In a first aspect, the present invention provides a genetically modified poppy plant of the species P. bracteatum, or a hybrid thereof, wherein the expression of one or more of thebaine 6-O-demethylase, codeine O-demethylase and/or codeinone reductase is increased relative to wild type P. bracteatum and wherein said genetically modified poppy plant produces an increased quantity of an alkaloid selected from codeine, oripavine and/or morphine relative to a wild type P. bracteatum.

Reference herein to a “poppy plant” may refer to a whole poppy plant, but may also refer to a part of a poppy plant, including, for example, reproductive material (such as seeds) derived from a poppy plant; a cell, tissue or organ derived from a poppy plant; and the like. Thus, in some embodiments, the present invention also provides poppy plant cells, tissues, organs, reproductive material and the like.

As set out above, the poppy plants of the present invention are of the perennial poppy species, Papaver bracteatum. Typical P. bracteatum, or Persian Poppy, grows to a height of up to 1.5 metres. It has 6 petals which are blood red in colour with a dark basal blotch. The capsules are typically about 40 mm diameter with a concave cap and persistent bracts. As would be appreciated by a person skilled in the art, the above morphological description may not apply to all members of the species and thus the morphological description should not be considered to limit the scope of the term P. bracteatum as used herein.

Furthermore, reference herein to a poppy plant of the present invention should be understood to include P. bracteatum species as well as hybrid plants wherein at least one of the parents is a plant of the species P. bracteatum. Such hybrids may include, for example, an intrageneric hybrid plant wherein one of the parents is a plant of the first aspect of the invention and the other parent is a plant within the genus Papaver (e.g. Papaver somniferum or Papaver orientale).

In some embodiments, the hybrid may be an intergeneric hybrid plant wherein one of the parents is a plant of the first aspect of the invention and the other parent is a plant of a genus other than Papaver.

As set out above, the present invention contemplates genetically modified poppy plants in which the expression of one or more of thebaine 6-O-demethylase, codeine O-demethylase and/or codeinone reductase is increased relative to wild type P. bracteatum. Reference herein to a “wild type P. bracteatum” should be understood to be native or non-genetically modified or non-hybridised plants of the species P. bracteatum.

As set out above, the genetically modified poppy plants of the present invention comprise increased expression of one or more of thebaine 6-O-demethylase (T6ODM), codeine O-demethylase (CODM) and/or codeinone reductase (COR) relative to wild type Papaver bracteatum.

The term “increased expression” as used herein is intended, for example to refer to a 1%, 5%, 10%, 20%, 30%, 40%, 50%, 60%, 70%, 80%, 90%, 100%, 2-fold, 5-fold, 10-fold, 20-fold, 50-fold, 100-fold or greater increase in the enzymatic activity of T6ODM, CODM and/or COR in the genetically modified poppy plants of the present invention relative to wild type P. bracteatum. In some embodiments, increased expression may comprise the introduction of T6ODM, CODM and/or COR activity into a poppy plant of the present invention where such activity is absent in wild type P. bracteatum.

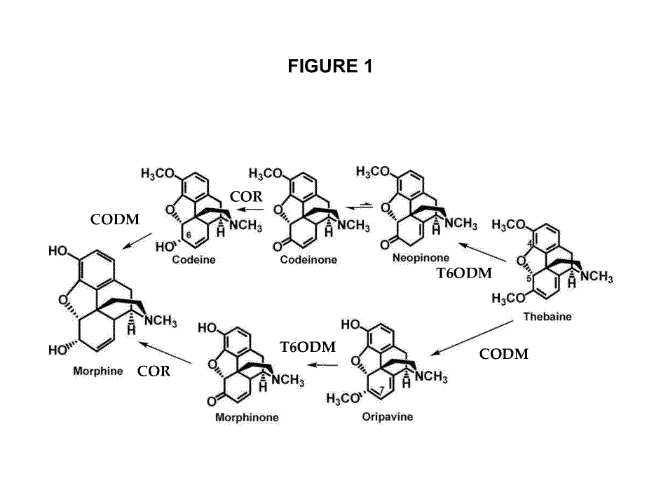

As shown in FIG. 1, in the synthesis of morphinane alkaloids, T6ODM catalyses the demethylation of thebaine to neopinone and also catalyses the demethylation of oripavine to morphinone. CODM catalyses the demethylation of thebaine to oripavine and also catalyses the demethylation of codeine to morphine. COR catalyses the reduction of codeinone to codeine and also catalyses the reduction of morphinone to morphine.

The present inventors have noted that wild-type P. bracteatum accumulates significant amounts of thebaine, while later the alkaloids codeine, oripavine and morphine are either absent, or at very low levels, in wild type P. bracteatum. As such, the inventors have determined that one or more of the biosynthetic enzymes responsible for the conversion of thebaine through to morphine (via codeine and/or oripavine) are either absent or disabled in wild type P. bracteatum.

Notwithstanding, the present inventors have also identified that in at least some cultivars of P. bracteatum or hybrids thereof low levels of oripavine may accumulate. Accordingly, in these cultivars, it has been determined that CODM may be active, at least at a low level. As such, in these plants, it may only be necessary to introduce T6ODM and, optionally, COR activity into the plant in order to produce morphine and/or codeine in the plant. However, even in such cultivars where CODM is active, it may be desirable to increase the activity of CODM in order to increase the yield of morphine and/or codeine in the plant. Alternatively, P. bracteatum cultivars with very low or absent expression of CODM may be beneficial where it is desirable to produce predominantly codeine in the plant and either low or no morphine in the plant.

As shown in Example 2, the present inventors have data to indicate that the COR gene is present in P. bracteatum. Furthermore, Brochmann-Hanssen & Wunderly (J Pharm Sci 67(1): 103-106, 1978) have reported that when codeinone was administered to living P. bracteatum, it was converted to codeine rapidly and efficiently. From this report, the present inventors have determined that in at least some P. bracteatum cultivars, COR may also be active. However, even in such cultivars where COR is active, it may be desirable to increase the activity of COR in order to increase the yield of morphine and/or codeine in the plant.

In light of the above, the present inventors have determined that in order to obtain production of an alkaloid selected from codeine, oripavine and/or morphine in P. bracteatum, the expression of one or more of T6ODM, CODM and/or COR activity may be increased in P. bracteatum.

In some embodiments, at least the expression of T6ODM is increased in the poppy plants of the present invention relative to wild type P. bracteatum.

The term “genetically modified poppy plant”, as used herein, should be understood to refer to a poppy plant that has had a genetic modification made to it. A “genetic modification” may include any genetic modification that effects an increase in the expression of an enzymatic activity of interest (eg. T6ODM, CODM and/or COR) in a genetically modified plant relative to a non-genetically modified form of the plant. Exemplary types of genetic modification include: random mutagenesis such as transposon, chemical, UV and phage mutagenesis together with selection of mutants which overexpress an enzymatic activity of interest; transient or stable introduction of one or more nucleic acid molecules into a cell which direct the expression and/or overexpression of an enzyme of interest in the cell; and the like.

In some embodiments, the present invention contemplates increasing the expression of T6ODM, CODM and/or COR in P. bracteatum, by increasing the expression of a T6ODM, CODM and/or COR encoding nucleic acid in one or more cells of the plant and/or increasing the copy number of a T6ODM, CODM and/or COR encoding nucleic acid in one or more cells of the plant.

By “increasing the expression of a T6ODM, CODM and/or COR encoding nucleic acid” is intended, for example a 1%, 5%, 10%, 20%, 30%, 40%, 50%, 60%, 70%, 80%, 90%, 100%, 2-fold, 5-fold, 10-fold, 20-fold, 50-fold, 100-fold or greater increase in the transcription and/or translation of a T6ODM, CODM and/or COR encoding nucleic acid relative to wild type P. bracteatum. Increasing the expression of a T6ODM, CODM and/or COR encoding nucleic acid should also be understood to comprise introducing the expression of a T6ODM, CODM and/or COR encoding nucleic acid into a poppy plant of the present invention where such activity is absent in wild type P. bracteatum.

Thus, in some embodiments, the genetically modified poppy plant of the present invention comprises one or more transgenes which encode T6ODM, CODM and/or COR. In some embodiments, the genetically modified poppy plant at least comprises one or more transgenes which encode thebaine 6-O-demethylase.

A “transgene” as referred to herein should be understood to include any nucleotide sequence which is introduced into and/or is non-native to the wild type P. bracteatum genome. As such, a transgene may be an additional or replacement copy of a nucleotide sequence already in the P. bracteatum genome or may be a nucleotide sequence which is exogenous to the wild type P. bracteatum genome.

Reference herein to “thebaine 6-O-demethylase” or “T6ODM” should be understood to include any enzyme which can catalyse the O-demethylation of thebaine to neopinone and/or the O-demethylation of oripavine to morphinone. As such, reference herein to a nucleotide sequence which encodes thebaine 6-O-demethylase should be understood to include any nucleotide sequence which encodes a thebaine 6-O-demethylase enzyme. An example of a nucleotide sequence which encodes thebaine 6-O-demethylase includes the nucleotide sequence set forth in GenBank accession number GQ500139 or a functional homolog or variant thereof.

Reference herein to “codeine O-demethylase” or “CODM” should be understood to include any enzyme which can catalyse the O-demethylation of thebaine to oripavine and/or the O-demethylation of codeine to morphine. As such, reference herein to a nucleotide sequence which encodes codeine O-demethylase should be understood to include any nucleotide sequence which encodes a codeine O-demethylase enzyme. An example of a nucleotide sequence which encodes codeine O-demethylase includes the nucleotide sequence set forth in GenBank accession number GQ500141 or a functional homolog or variant thereof.

Reference herein to “codeinone reductase” or “COR” should be understood to include any enzyme which can catalyse the reduction of codeinone to codeine and/or the reduction of morphinone to morphine. As such, reference herein to a nucleotide sequence which encodes codeinone reductase should be understood to include any nucleotide sequence which encodes a codeinone reductase enzyme. An example of a nucleotide sequence which encodes codeinone reductase includes the nucleotide sequence set forth in GenBank accession number FJ596162 or a functional homolog or variant thereof.

Reference herein to a “functional homolog or variant” of a particular reference sequence (e.g. the nucleotide sequence set forth in any of GenBank accession numbers GQ500139, GQ500141 or FJ596162) may be a nucleic acid which has one or more nucleotide insertions, deletions or substitutions relative to the reference sequence; a mutant form or allelic variant of the reference sequence; an ortholog of the reference sequence; analogs that contain one or more modified bases or DNA or RNA backbones modified for stability or for other reasons, and the like. “Modified” bases include, for example, tritylated and unusual bases such as inosine. The functional homolog or variant retains the ability to encode an enzyme having equivalent activity to the reference sequence.

In some embodiments, a functional homolog or variant comprises at least 50% sequence identity to the reference nucleotide sequence, including at least 55% nucleotide sequence identity, at least 60% nucleotide sequence identity, at least 65% nucleotide sequence identity, at least 70% nucleotide sequence identity, at least 75% nucleotide sequence identity, at least 80% nucleotide sequence identity, at least 85% nucleotide sequence identity, at least 90% nucleotide sequence identity or at least 95%, 96%, 97%, 98%, 99% or 100% nucleotide sequence identity to the reference nucleotide sequence.

For example, the inventors have identified variants of the reference sequences for T6ODM and CODM. With respect to T6ODM, four variants have been identified which respectively comprise the cDNA nucleotide sequences set forth in SEQ ID NOs: 1 to 4. These nucleotide sequences are derived from genomic DNA clones comprising the nucleotide sequences set forth in SEQ ID NOs: 5 to 8, respectively. The identified T6ODM nucleotide sequence variants collectively encode T6ODM enzyme variants comprising the amino acid sequences set forth in SEQ ID NOs: 9 to 11.

With respect to CODM, a single nucleotide sequence variant has been identified which comprises the cDNA nucleotide sequence set forth in SEQ ID NO: 12. This nucleotide sequence is derived from a genomic DNA clone comprising the nucleotide sequence set forth in SEQ ID NO: 13. The identified CODM variant does not encode a variant CODM polypeptide.

In the case of COR-encoding nucleotide sequences, a number of functional homologs or variants of the nucleotide sequence set forth in FJ596162 (which encode COR enzymes) are known in the art. For example, the following GenBank accession numbers are nucleotide sequences which have been reported to encode COR: FJ596169.1, FJ596168.1, FJ596167.1, FJ596166.1, FJ596165.1, FJ596163.1, FJ596161.1, FJ596160.1, NC_{—}008403.2, NC_{—}008397.2, NC_{—}008396.2, NM_{—}001070581.1, NM_{—}001070580.1, NM_{—}001059452.1, NM_{—}001059451.1, NM_{—}001056028.1, AF108435.1, AF108434.1, AF108433.1, AF108432.1, AF108437.1 and FJ624147.1.

When comparing nucleotide sequences to calculate a percent identity, the compared sequences should be compared over a comparison window of at least 100 nucleotide residues, at least 200 nucleotide residues, at least 300 nucleotide residues, at least 400 nucleotide residues, at least 500 nucleotide residues, at least 600 nucleotide residues, at least 800 nucleotide residues, at least 1000 nucleotide residues, or over the full length of the reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerised implementations of algorithms such as the BLAST family of programs as, for example, disclosed by Altschul et al. 1997 (Nucl. Acids Res. 25: 3389-3402). Global alignment programs may also be used to align similar sequences of roughly equal size. Examples of global alignment programs include NEEDLE (available at www.ebi.ac.uk/Tools/psa/emboss_needle/) which is part of the EMBOSS package (Rice P et al., 2000, Trends Genet., 16: 276-277), and the GGSEARCH program (available at fasta.bioch.virginia.edu/fasta_www2/fasta_www.cgi?rm=compare&pgm=gnw) which is part of the FASTA package (Pearson W and Lipman D, 1988, Proc. Natl. Acad. Sci. USA, 85: 2444-2448). Both of these programs are based on the Needleman-Wunsch algorithm which is used to find the optimum alignment (including gaps) of two sequences along their entire length. A detailed discussion of sequence analysis can also be found in Unit 19.3 of Ausubel et al (“Current Protocols in Molecular Biology” John Wiley & Sons Inc, 1994-1998, Chapter 15, 1998). In some embodiments, only the protein coding regions of the functional homolog and reference sequence may be compared.

In order to effect expression of a transgene in one or more cells of a genetically modified plant, where appropriate, the transgene may be operably connected to one or more transcriptional control sequences and/or promoters. In some embodiments, a T6ODM, CODM and/or COR encoding transgene may be operably connected to a transcriptional control sequence such as a native T6ODM, CODM and/or COR promoter or a heterologous promoter.

For the purposes of the present specification, a transcriptional control sequence is regarded as “operably connected” to a given gene or other nucleotide sequence when the transcriptional control sequence is able to promote, inhibit or otherwise modulate the transcription of the gene or other nucleotide sequence.

A promoter may regulate the expression of an operably connected nucleotide sequence constitutively, or differentially, with respect to the cell, tissue, organ or developmental stage at which expression occurs, in response to external stimuli such as physiological stresses, pathogens, or metal ions, amongst others, or in response to one or more transcriptional activators. As such, the promoter used in accordance with the methods of the present invention may include, for example, a constitutive promoter, an inducible promoter, a tissue-specific promoter or an activatable promoter.

Plant constitutive promoters typically direct expression in nearly all tissues of a plant and are largely independent of environmental and developmental factors. Examples of constitutive promoters that may be used in accordance with the present invention include plant viral derived promoters such as the Cauliflower Mosaic Virus 35S and 19S (CaMV 35S and CaMV 19S) promoters; bacterial plant pathogen derived promoters such as opine promoters derived from Agrobacterium spp., e.g. the Agrobacterium-derived nopaline synthase (nos) promoter; and plant-derived promoters such as the rubisco small subunit gene (rbcS) promoter, the plant ubiquitin promoter (Pubs) and the rice actin promoter (Pact).

Furthermore, variations of the aforementioned promoters may be used. For example a CaMV 35S promoter may be used that comprises dual enhancer motifs (see Example 4). Such a promoter is represented by the sequence set forth in SEQ ID NO: 14.

“Inducible” promoters include, but are not limited to, chemically inducible promoters and physically inducible promoters. Chemically inducible promoters include promoters which have activity that is regulated by chemical compounds such as alcohols, antibiotics, steroids, metal ions or other compounds. Examples of chemically inducible promoters include: alcohol regulated promoters (eg. see European Patent 637 339); tetracycline regulated promoters (eg. see U.S. Pat. No. 5,851,796 and U.S. Pat. No. 5,464,758); steroid responsive promoters such as glucocorticoid receptor promoters (eg. see U.S. Pat. No. 5,512,483), estrogen receptor promoters (eg. see European Patent Application 1 232 273), ecdysone receptor promoters (eg. see U.S. Pat. No. 6,379,945) and the like; metal-responsive promoters such as metallothionein promoters (eg. see U.S. Pat. No. 4,940,661, U.S. Pat. No. 4,579,821 and U.S. Pat. No. 4,601,978); and pathogenesis related promoters such as chitinase or lysozyme promoters (eg. see U.S. Pat. No. 5,654,414) or PR protein promoters (eg. see U.S. Pat. No. 5,689,044, U.S. Pat. No. 5,789,214, Australian Patent 708850, U.S. Pat. No. 6,429,362).

The inducible promoter may also be a physically regulated promoter which is regulated by non-chemical environmental factors such as temperature (both heat and cold), light and the like. Examples of physically regulated promoters include heat shock promoters (eg. see U.S. Pat. No. 5,447,858, Australian Patent 732872, Canadian Patent Application 1324097); cold inducible promoters (eg. see U.S. Pat. No. 6,479,260, U.S. Pat. No. 6,184,443 and U.S. Pat. No. 5,847,102); light inducible promoters (eg. see U.S. Pat. No. 5,750,385 and Canadian Patent 132 1563); light repressible promoters (eg. see New Zealand Patent 508103 and U.S. Pat. No. 5,639,952).

“Tissue specific promoters” include promoters which are preferentially or specifically expressed in one or more specific cells, tissues or organs in an organism and/or one or more developmental stages of the organism. It should be understood that a tissue specific promoter may also be inducible.

Examples of plant tissue specific promoters include: root specific promoters such as those described in US patent application 21047525; fruit specific promoters including ovary specific and receptacle tissue specific promoters such as those described in European Patent 316 441, U.S. Pat. No. 5,753,475 and European Patent Application 973 922; and seed specific promoters such as those described in Australian Patent 612326 and European Patent application 0 781 849 and Australian Patent 746032.

The promoter may also be a promoter that is activatable by one or more transcriptional activators, referred to herein as an “activatable promoter”. For example, the activatable promoter may comprise a minimal promoter operably connected to an Upstream Activating Sequence (UAS), which comprises, inter alia, a DNA binding site for one or more transcriptional activators.

As referred to herein the term “minimal promoter” should be understood to include any promoter that incorporates at least an RNA polymerase binding site and, optionally a TATA box and transcription initiation site and/or one or more CAAT boxes. In some embodiments wherein the cell is a plant cell, the minimal promoter may be derived from the Cauliflower Mosaic Virus 35S (CaMV 35S) promoter. The CaMV 35S derived minimal promoter may comprise, for example, a sequence that substantially corresponds to positions −90 to +1 (the transcription initiation site) of the CaMV 35S promoter (also referred to as a −90 CaMV 35S minimal promoter), −60 to +1 of the CaMV 35S promoter (also referred to as a −60 CaMV 35S minimal promoter) or −45 to +1 of the CaMV 35S promoter (also referred to as a −45 CaMV 35S minimal promoter).

As set out above, the activatable promoter may comprise a minimal promoter fused to an Upstream Activating Sequence (UAS). The UAS may be any sequence that can bind a transcriptional activator to activate the minimal promoter. Exemplary transcriptional activators include, for example: yeast derived transcription activators such as Gal4, Pdr1, Gcn4 and Ace1; the viral derived transcription activator, VP16; Hap1 (Hach et al., J Biol Chem 278: 248-254, 2000); Gaf1 (Hoe et al., Gene 215(2): 319-328, 1998); E2F (Albani et al., J Biol Chem 275: 19258-19267, 2000); HAND2 (Dai and Cserjesi, J Biol Chem 277: 12604-12612, 2002); NRF-1 and EWG (Herzig et al., J Cell Sci 113: 4263-4273, 2000); P/CAF (Itoh et al., Nucl Acids Res 28: 4291-4298, 2000); MafA (Kataoka et al., J Biol Chem 277: 49903-49910, 2002); human activating transcription factor 4 (Liang and Hai, J Biol Chem 272: 24088-24095, 1997); Bcl10 (Liu et al., Biochem Biophys Res Comm 320(1): 1-6, 2004); CREB-H (Omori et al., Nucl Acids Res 29: 2154-2162, 2001); ARR1 and ARR2 (Sakai et al., Plant J 24(6): 703-711, 2000); Fos (Szuts and Bienz, Proc Natl Acad Sci USA 97: 5351-5356, 2000); HSF4 (Tanabe et al., J Biol Chem 274: 27845-27856, 1999); MAML1 (Wu et al., Nat Genet 26: 484-489, 2000).

In some embodiments, the UAS comprises a nucleotide sequence that is able to bind to at least the DNA-binding domain of the GAL4 transcriptional activator. UAS sequences, which can bind transcriptional activators that comprise at least the GAL4 DNA binding domain, are referred to herein as UAS_{G}.

The UAS sequence in the activatable promoter may comprise a plurality of tandem repeats of a DNA binding domain target sequence. For example, in its native state, UAS_{G} comprises four tandem repeats of the DNA binding domain target sequence. As such, the term “plurality” as used herein with regard to the number of tandem repeats of a DNA binding domain target sequence should be understood to include, for example, at least 2 tandem repeats, at least 3 tandem repeats or at least 4 tandem repeats.

The transcriptional control sequence may also include a terminator. The term “terminator” refers to a DNA sequence at the end of a transcriptional unit which signals termination of transcription. Terminators are 3′-non-translated DNA sequences generally containing a polyadenylation signal, which facilitate the addition of polyadenylate sequences to the 3′-end of a primary transcript. As with promoter sequences, the terminator may be any terminator sequence which is operable in the cells, tissues or organs in which it is intended to be used. Examples of suitable terminator sequences which may be useful in plant cells include: the nopaline synthase (nos) terminator, the CaMV 35S terminator, the octopine synthase (ocs) terminator, potato proteinase inhibitor gene (pin) terminators, such as the pinII and pinIII terminators and the like.

The transcriptional control sequence to which a transgene is connected may be introduced into a cell with the transgene, or alternatively, the transgene may be inserted into the genome of the plant cell such that it becomes operably connected to an endogenous transcriptional control sequence. In the latter embodiments, the insertion of the transgene in the genome such that it is under the control of an endogenous transcriptional control sequence may be the result of either non-site directed or random DNA insertion (eg. T-DNA or transposon mediated insertion) or the result of site-directed insertion (for example as described in Terada et al., Nat. Biotechnol. 20: 1030-1034, 2002).

The one or more transgenes may be contained within a vector or construct for transformation into P. bracteatum. Where the vector or construct comprises the one or more transgenes operably connected to transcriptional control sequences which are active in P. bracteatum, the vector or construct may be referred to as an expression vector or construct.

The vector or construct may further comprise, for example, one or more of: an origin of replication for one or more hosts (including bacteria such as E. coli or Agrobacterium spp.); a selectable marker gene which is active in one or more hosts; and/or one or more additional transcriptional control sequences.

As used herein, the term “selectable marker gene” includes any gene that confers a phenotype on a cell in which it is expressed, to facilitate the identification and/or selection of cells which are transfected or transformed with a genetic construct of the invention.

“Selectable marker genes” include any nucleotide sequences which, when expressed by a cell, confer a phenotype on the cell that facilitates the identification and/or selection of these transformed cells. A range of nucleotide sequences encoding suitable selectable markers are known in the art. Exemplary nucleotide sequences that encode selectable markers include: antibiotic resistance genes such as ampicillin-resistance genes, tetracycline-resistance genes, kanamycin-resistance genes, the AURI-C gene which confers resistance to the antibiotic aureobasidin A, neomycin phosphotransferase genes (eg. nptI and nptII) and hygromycin phosphotransferase genes (eg. hpt); herbicide resistance genes including glufosinate, phosphinothricin or bialaphos resistance genes such as phosphinothricin acetyl transferase encoding genes (eg. bar), glyphosate resistance genes including 3-enoyl pyruvyl shikimate 5-phosphate synthase encoding genes (eg. aroA), bromyxnil resistance genes including bromyxnil nitrilase encoding genes, sulfonamide resistance genes including dihydropterate synthase encoding genes (eg. sul) and sulfonylurea resistance genes including acetolactate synthase encoding genes; enzyme-encoding reporter genes such as GUS and chloramphenicolacetyltransferase (CAT) encoding genes; fluorescent reporter genes such as the green fluorescent protein-encoding gene; and luminescence-based reporter genes such as the luciferase gene, amongst others.

In some embodiments, the vector or construct is adapted to be at least partially transferred into a plant cell via Agrobacterium-mediated transformation. Accordingly, in some embodiments, the construct may comprise left and/or right T-DNA border sequences.

Suitable T-DNA border sequences would be readily ascertained by one of skill in the art. However, the term “T-DNA border sequences” should be understood to include, for example, any substantially homologous and substantially directly repeated nucleotide sequences that delimit a nucleic acid molecule that is transferred from an Agrobacterium sp. cell into a plant cell susceptible to Agrobacterium-mediated transformation. By way of example, reference is made to the paper of Peralta and Ream (Proc. Natl. Acad. Sci. USA, 82(15): 5112-5116, 1985) and the review of Gelvin (Microbiology and Molecular Biology Reviews, 67(1): 16-37, 2003).

In some embodiments, the vector or construct is adapted to be transferred into a plant via Agrobacterium-mediated transformation, however, the present invention also contemplates any suitable modifications to the genetic construct that facilitate bacterial mediated insertion into a plant cell via bacteria other than Agrobacterium sp., for example as described in Broothaerts et al. (Nature 433: 629-633, 2005).

Those skilled in the art will be aware of how to produce the constructs described herein and of the requirements for obtaining the expression thereof, when so desired, in a specific cell or cell-type under the conditions desired. In particular, it will be known to those skilled in the art that the genetic manipulations required to perform the present invention may require the propagation of a genetic construct described herein or a derivative thereof in a prokaryotic cell such as an E. coli cell or a plant cell or an animal cell. Exemplary methods for cloning nucleic acid molecules are described in Sambrook et al. (Molecular Cloning—A Laboratory Manual, 3^{rd} Ed., Cold Spring Harbor Laboratory Press, 2000).

As such, in another aspect, the present invention provides an expression construct comprising one or more one or more transgenes which encode thebaine 6-O-demthylase, codeine O-demethylase and/or codeinone reductase wherein said one or more transgenes are operably connected to a transcriptional control sequence which is active in P. bracteatum.

In order to effect expression of a transgene in P. bracteatum, a transgene or a construct including a transgene must be introduced into P. bracteatum via a suitable transformation method.

Methods for the transformation of plant cells include, for example: Agrobacterium-mediated transformation, microprojectile bombardment based transformation methods and direct DNA uptake based methods. Roa-Rodriguez et al. (Agrobacterium-mediated transformation of plants, 3^{rd} Ed. CAMBIA Intellectual Property Resource, Canberra, Australia, 2003) review a wide array of suitable Agrobacterium-mediated plant transformation methods for a wide range of plant species. Bacterial mediated transformation using bacteria other than Agrobacterium sp. may also be used, for example as described in Broothaerts et al. (Nature 433: 629-633, 2005). Microprojectile bombardment may also be used to transform plant tissue and methods for the transformation of plants, including cereal plants, and such methods are reviewed by Casas et al. (Plant Breeding Rev. 13: 235-264, 1995). Direct DNA uptake transformation protocols such as protoplast transformation and electroporation are described in detail in Galbraith et al. (eds.), Methods in Cell Biology Vol. 50, Academic Press, San Diego, 1995). In addition to the methods mentioned above, a range of other transformation protocols may also be used. These include infiltration, electroporation of cells and tissues, electroporation of embryos, microinjection, pollen-tube pathway, silicon carbide- and liposome mediated transformation. Methods such as these are reviewed by Rakoczy-Trojanowska (Cell. Mol. Biol. Lett. 7: 849-858, 2002). A range of other plant transformation methods may also be evident to those of skill in the art. By way of further example, reference is also made to Zhao et al. (Mol Breeding DOI 10.1007/s11032-006-9005-6, 2006), Katsuhara et al. (Plant Cell Physiol 44(12): 1378-1383, 2003), Ohta et al. (FESS Letters 532: 279-282, 2002) and Wu et al. (Plant Science 169: 65-73, 2005).

In some embodiments, the method of Solouki et al. (Trakia Journal of Sciences 7(2): 1-7, 2009) is an example of a method known to be suitable for the transformation of P. bracteatum.

In further embodiments, P. bracteatum may be transformed according to methods set out in the Examples (see later).

As set out above, the genetically modified poppy plants of the present invention produce an increased quantity of an alkaloid selected from codeine, oripavine and/or morphine relative to a wild type P. bracteatum.

The “increased quantity of codeine, oripavine and/or morphine” may occur in one or more tissues of the genetically modified poppy plant, or in the latex of the genetically modified poppy plant, relative to wild-type P. bracteatum. For example, in some embodiments the increased quantity of codeine, oripavine and/or morphine in the genetically modified poppy plants of the present invention may occur in the latex and/or one or more tissues selected from root tissue, leaf tissue and/or capsule.

By “increased quantity” is intended for example, to refer to a 1%, 5%, 10%, 20%, 30%, 40%, 50%, 60%, 70%, 80%, 90%, 100%, 2-fold, 5-fold, 10-fold, 20-fold, 50-fold, 100-fold or greater increase in the quantity of codeine, oripavine and/or morphine in the poppy plants of the present invention relative to wild type P. bracteatum.

In some embodiments, the poppy plants of the present invention produce an extractable quantity of codeine, oripavine and/or morphine. An “extractable quantity” of codeine, oripavine and/or morphine should be understood to include an amount of codeine, oripavine and/or morphine which is extractable and detectable from one or more tissues of the plant. The one or more tissues may be selected from those referred to above. In some embodiments, an “extractable quantity” of codeine, oripavine and/or morphine should be understood to include wherein any of codeine, oripavine and/or morphine, or a combination of codeine, oripavine and/or morphine, represents at least 1%, at least 5%, at least 10%, at least 20%, at least 30%, at least 40%, at least 50%, at least 60%, at least 70%, at least 80% or at least 90% by weight of the total extractable alkaloid from any one or more of the latex, capsules, roots or leaves of the poppy plants of the present invention.

As set out above, the genetically modified poppy plants contemplated by the present invention are typically perennial. As referred to herein, a “perennial” poppy plant encompasses poppy plants that produce seed capsules and an increased and/or extractable quantity of an alkaloid (such as codeine, oripavine and/or morphine) for more than one growing season without the need to replant.

In some embodiments, the poppy plants of the present invention produce seed capsules and an increased and/or extractable quantity of codeine, oripavine and/or morphine in the first growing season after germination in a temperate poppy growing region.

Production of seed capsules and an increased and/or extractable quantity of codeine, oripavine and/or morphine “in the first growing season after germination” should be understood as the plants producing seed capsules and an increased and/or extractable quantity of codeine, oripavine and/or morphine within one year of seed germination.

As referred to herein, “a temperate poppy growing region” should be understood as a region in which poppy plants may be cultivated which is located at a latitude that lies between the tropics and the polar circles. Specifically, the north temperate zone extends from the Tropic of Cancer at about 23.5 degrees north latitude to the Arctic Circle at about 66.5 degrees north latitude. The south temperate zone extends from the Tropic of Capricorn at about 23.5 degrees south latitude to the Antarctic Circle at about 66.5 degrees south Latitude.

In some embodiments, the temperate poppy growing region may include a region where the poppy plants of the present invention will grow which is at one or more North or South latitudes of about 34°, about 35°, about 36°, about 37°, about 38°, about 39°, about 40°, about 41°, about 42°, about 43°, about 44°, about 45°, about 46°, about 47°, about 48°, about 49°, and/or about 50°.

In some embodiments, the poppy plants of the present invention produce seed capsules and an increased and/or extractable quantity of codeine, oripavine and/or morphine in the first growing season after germination in at least a temperate poppy growing region in the Southern hemisphere. Thus, in some embodiments, the poppy plants of the present invention may produce seed capsules and an increased and/or extractable quantity of codeine, oripavine and/or morphine in the first growing season after germination at one or more latitudes of about 34° S, about 35° S, about 36° S, about 37° S, about 38° S, about 39° S, about 40° S, about 41° S, about 42° S, about 43° S, about 44° S, about 45° S, about 46° S, about 47° S, about 48° S, about 49° S, and/or about 50° S.

Reference herein to a latitude “about” a particular latitude should be understood to encompass latitudes ±0.5° of the defined latitude. In addition, reference to “one or more latitudes” should be understood to encompass discreet ranges of latitudes bounded by any pairs of the specific latitudes mentioned above.

In some embodiments, the poppy plants of the present invention produce seed capsules and an increased and/or extractable quantity of codeine, oripavine and/or morphine in the first growing season after germination at a latitude of between about 40° S and about 44° S.

In some embodiments, the poppy plants of the present invention produce seed capsules and an increased and/or extractable quantity of codeine, oripavine and/or morphine in the first growing season after germination in Tasmania, Australia or a region having substantially equivalent climate.

The average rainfall and temperatures in Tasmania vary considerably across the state. However, in the main poppy growing regions in the north-west and midlands of the state, the typical rainfall is in the range of 600 to 800 mm, and the minimum/maximum temperatures are 2° C. and 12° C. respectively for July and 12° C. and 24° C. for January and February. Typically, in Tasmanian poppy growing regions, poppies are planted in March and the dried poppy heads are harvested in the following February.

Reference herein to a “region having substantially equivalent climate” refers to a region having equivalent day length, rainfall and/or seasonal temperature minima and maxima such that conditions under which poppies may be grown are equivalent to those in poppy growing regions of Tasmania, Australia.

The poppy plants of the present invention may have either a dehiscent (open) seed capsule or an indehiscent (closed) seed capsule at seed maturity.

Wild type P. bracteatum, and most other Papaver species, form pores of dehiscence in the seed capsule at seed maturity, which allows the mature seed in the seed capsule to be released from the seed capsule. However, in some embodiments of the present invention, the poppy cultivar may have seed capsules that have reduced or no pores of dehiscence at the time the seed in the seed capsule has matured. Thus, the trait of an indehiscent or closed seed capsule at maturity leads to the retention of mature seed in the seed capsule in cultivars having the trait.

One significant advantage of the indehiscent or closed seed capsule at maturity trait is that seed is retained on harvesting, and the dispersal of seed is substantially more controlled, minimising excessive plant density with perennial species. Additionally, the seed from cultivars having an indehiscent seed capsule can be readily collected from the seed capsule and further utilised.

The present invention also provides a progeny plant, wherein the progeny plant has a plant of the first aspect of the invention as a parent, wherein the progeny plant comprises increased expression of one or more of thebaine 6-O-demethylase, codeine O-demethylase and/or codeinone reductase relative to wild type P. bracteatum, and wherein said progeny plant produces an increased and/or extractable quantity of an alkaloid selected from codeine, oripavine and/or morphine relative to a wild type P. bracteatum.

As referred to herein, a “progeny” plant may be any plant for which a plant of the first aspect of the invention is at least one of the parents. Thus, the progeny plant may be a plant resulting from self-fertilisation of a plant of the first aspect of the invention; a plant resulting from a non-self fertilisation wherein a plant of the first aspect of the invention is the male or female parent; an intrageneric hybrid plant wherein one of the parents is a plant of the first aspect of the invention and the other parent is a plant within the genus Papaver (e.g. Papaver somniferum or Papaver orientale); an intergeneric hybrid plant wherein one of the parents is a plant of the first aspect of the invention and the other parent is a plant of a genus other than Papaver; an asexually produced progeny of a plant of the first aspect of the invention, such as vegetatively reproduced progeny or progeny produced by apomixis.

In another aspect, the present invention also provides a mutant or derivative plant of the poppy plant of the first aspect of the invention wherein the mutant or derivative comprises increased expression of one or more of thebaine 6-O-demethylase, codeine O-demethylase and/or codeinone reductase relative to wild type P. bracteatum, and wherein said mutant or derivative produces an increased and/or extractable quantity of an alkaloid selected from codeine, oripavine and/or morphine relative to a wild type P. bracteatum.

A “mutant or derivative” of the subject poppy plants should be understood to encompass, for example, any spontaneous or induced mutant, breeding progeny or further genetically modified forms of the poppy plants of the first aspect of the invention.

Mutagenisation techniques that may be used to generate derivatives or mutants of the poppy plants of the present invention include, for example, chemical mutagenesis (eg. EMS mutagenesis), ionising radiation-induced mutagenesis (eg. X-ray mutagenesis, γ-ray mutagenesis and UV mutagenesis), genetic insertion mutagenesis methods (eg. transposon mutagenesis or T-DNA mutagenesis) and the like. Techniques for the production of mutagenized seed are well known in the art. For example, methods of seed mutagenesis as well as chemical mutagens suitable for use in these methods are described in, for example, The Manual on Mutation Breeding, 2nd ed. (I.A.E.A., Vienna, 1977) or in Plant Breeding, Principles and Prospects (Chapman and Hall, London, 1993). Example mutagens include ethyl methanesulfonate (EMS), diepoxybutane (DEB) ethyl-2-chloroethyl sulphide, 2-chloroethyl-dimethylamine, ethylene oxide, ethyleneimine, dimethyl sulphonate, diethyl sulphonate, propane sulphone, beta-propiolactone, diazomethane, N-methyl-N-nitrosourethane, acridine orange and sodium azide. An example of ionising radiation induced mutagenesis, specifically X-ray mutagenesis is described and compared to EMS mutagenesis by Filippetti et al. (Euphytica 35: 49-59, 1986).

In some embodiments, the progeny, mutant or derivative plants of the present invention produce seed capsules and an increased and/or extractable quantity of codeine, oripavine and/or morphine in the first growing season after germination in one or more of the geographical locations hereinbefore described.

The present invention also provides reproductive material derived from the plants described herein. The term “reproductive material” should be understood as any material from which a plant may be reproduced. For example, “reproductive material” may include seeds, flowers, cuttings, ovaries, ovules, embryo sacs, egg cells, anthers, pollen, regenerable de-differentiated plant tissue such as callus, embryogenic callus or suspension culture, isolated plant embryos and the like.

In some embodiments, the reproductive material comprises a seed. As referred to herein, a plant “seed” should be understood to refer to a mature or immature plant seed. As such, the term “seed” includes, for example, immature seed carried by a maternal plant or seed released from the maternal plant. The term “seed” should also be understood to include any seed plant sporophyte between the developmental stages of fertilisation and germination. However, in some embodiments, the term seed refers to a mature plant seed.

In another aspect, the present invention also provides straw produced from a plant of the first aspect of the invention, or a progeny, mutant or derivative thereof.

As defined herein the “straw” of a poppy plant includes fresh or dried tissue of a poppy plant. This tissue may include all or part of the plant, such as root tissue, shoot tissue, floral tissue or a seed capsule. In some embodiments, poppy “straw” includes fresh or dried poppy plant tissue which includes a mature seed capsule. The contemplated seed capsule may include seeds, or may be a capsule in which the seeds have been removed.

Methods for poppy straw processing to obtain alkaloids are well known in the art. However, by way of example, reference is made to French patent 748308 which discloses one example of a method for alkaloid extraction from poppy straw.

A solvent (for example, water or a super critical fluid, such as supercritical CO₂) may also be used to extract alkaloids from straw. Extracts produced according to such methods include “straw concentrates”.

Thus, in another aspect, the present invention also provides a poppy straw concentrate produced from a plant of the first aspect of the invention or a progeny, mutant or derivative thereof.

The term “poppy straw concentrate” should be understood to include any material arising when poppy straw has entered into a process for the concentration of its alkaloids. Not inconsistent with the foregoing definition, “poppy straw concentrates” should also be understood to include any crude or purified extracts of poppy straw in either liquid, solid or powder form which contain one or more phenanthrene alkaloids of an opium poppy.

When in liquid form, poppy straw concentrates may be further concentrated from a crude extract. Such concentrates may be either liquid concentrates, wherein a portion of the solvent has been removed, or powder form concentrates which result from removing substantially all of the solvent used for extraction of the poppy straw.

A poppy straw concentrate, as defined herein, may include all of the alkaloids that may be extracted from a poppy straw, or may include a subset of the extractable alkaloids. Generally, a poppy straw concentrate of the present invention at least includes morphine and/or codeine.

In another aspect, the present invention also provides latex derived from the plant of the first aspect of the invention or a progeny, mutant or derivative thereof.

Methods for obtaining latex from a poppy plant, including P. bracteatum, are well known in the art. Generally, latex may be obtained by incising an immature seed capsule of the plant, from which latex is exuded.

In another aspect, the present invention also provides a stand of stably reproducing poppy plants, the stand comprising one or more plants according to the first aspect of the invention, or a progeny, mutant or derivative thereof.

In a yet further aspect, the present invention provides an isolated cell, tissue or organ derived from the plant of the first aspect of the invention or a progeny plant thereof.

The present invention also provides an in-vitro culture including one or more of the cells described above. Exemplary “in-vitro cultures” contemplated herein include, for example, callus cultures, embryogenic callus cultures, embryo cultures, plantlet cultures and suspension cultures which include one or more of the cells of the invention. Techniques for the establishment and maintenance of plant cell or tissue cultures are well known in the art. In this regard, reference is made to ‘Plant Tissue Culture: An Alternative for Production of Useful Metabolites’ (FAO Agricultural Services Bulletin No. 108, Food and Agriculture Organization of the United Nations Rome, 1994).

In some embodiments, an in-vitro culture of P. bracteatum may be established using the method described in U.S. Pat. No. 4,114,314, with the modification that cells derived from the plants of the present invention are used instead of those disclosed in U.S. Pat. No. 4,114,314.

In some embodiments, the in-vitro cultures of the present invention may be used to produce codeine, oripavine and/or morphine. The production of codeine, oripavine and/or morphine by the culture may be de novo production, or may be via the conversion of a complex substrate, such as an intermediate in the morphine or codeine biosynthetic pathway.

In another aspect, the present invention also provides a method of producing an alkaloid selected from codeine, oripavine and/or morphine, the method comprising growing a poppy plant of the first aspect of the invention, or a progeny, mutant or derivative thereof, such that the plant produces an alkaloid selected from codeine, oripavine and/or morphine; and extracting the codeine, oripavine and/or morphine from the poppy plant or a part thereof, or a progeny, mutant or derivative thereof.

In various embodiments, the method of the present invention comprises a method for producing codeine, oripavine and/or morphine in one or more of the geographical locations hereinbefore described.

In a further aspect, the present invention also provides codeine, oripavine and/or morphine produced according to the method above.

As indicated above, the inventors have identified nucleotide sequence variants of the T6ODM and CODM enzymes. Accordingly, in a further aspect, the present invention provides an isolated nucleic acid molecule comprising the nucleotide sequence set forth in any one of SEQ ID NOs: 1 to 8, 12 and 13.

In some embodiments, the present invention also provides T6ODM and CODM nucleotide sequence variants which display at least 50%, at least 55%, at least 60%, at least 65%, at least 70%, at least 75%, at least 80%, at least 85%, at least 90%, at least 91%, at least 92%, at least 93%, at least 94%, at least 95% at least 96%, at least 97%, at least 98%, at least 99% or 100% sequence identity to a nucleotide sequence set forth in any one of SEQ ID NOs: 1 to 8, 12 and 13.

In some embodiments, the T6ODM and CODM nucleotide sequence variants provided by the aspect of the present invention above do not consist of the cDNA sequences for T6ODM and CODM set forth in GenBank accessions GQ500139 and GQ500141, respectively.

When comparing nucleotide sequences to calculate a percentage identity, the compared sequences should be compared over a comparison window of at least 100 nucleotide residues, at least 200 nucleotide residues, at least 400 nucleotide residues, at least 600 nucleotide residues or over the full length of the relevant one of SEQ ID NOs: 1 to 8, 12 and 13. The comparison window may comprise additions or deletions (ie. gaps) of about 20% or less as compared to the reference sequence (which does not comprise additions or deletions) for optimal alignment of the two sequences. Optimal alignment of sequences for aligning a comparison window may be conducted by computerised implementations of algorithms such as the BLAST family of programs as, for example, disclosed by Altschul et al. (Nucl. Acids Res. 25: 3389-3402, 1997). A detailed discussion of sequence analysis can be found in Unit 19.3 of Ausubel et al. (“Current Protocols in Molecular Biology” John Wiley & Sons Inc, 1994-1998, Chapter 15, 1998).

In some embodiments, the present invention provides a nucleotide sequence which hybridises to a nucleic acid comprising the nucleotide sequence set forth in any one of SEQ ID NOs: 1 to 8, 12 and 13 under stringent conditions.

As used herein, “stringent” hybridisation conditions will be those in which the salt concentration is less than about 1.5 M Na ion, typically about 0.01 to 1.0 M Na ion concentration (or other salts) at pH 7.0 to 8.3 and the temperature is at least 30° C. Stringent conditions may also be achieved with the addition of destabilising agents such as formamide. In some embodiments, stringent hybridisation conditions may be low stringency conditions, medium stringency conditions or high stringency conditions. Exemplary low stringency conditions include hybridisation with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridisation in 40 to 45% formamide, 1.0 M NaCl, 1% SDS at 37° C., and a wash in 0.5× to 1×SSC at 55 to 60° C. Exemplary high stringency conditions include hybridisation in 50% formamide, 1 M NaCl, 1% SDS at 37° C., and a wash in 0.1×SSC at 60 to 65° C. Optionally, wash buffers may comprise about 0.1% to about 1% SDS. Duration of hybridisation is generally less than about 24 hours, usually about 4 to about 12 hours.

Specificity of hybridisation is also a function of post-hybridisation washes, with the critical factors being the ionic strength and temperature of the final wash solution. For DNA-DNA hybrids, the thermal melting point (Tₘ) can be approximated from the equation of Meinkoth and Wahl (Anal. Biochem. 138: 267-284, 1984), ie. Tₘ=81.5° C. +16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridisation solution, and L is the length of the hybrid in base pairs. The Tₘ is the temperature (under defined ionic strength and pH) at which 50% of a complementary target sequence hybridises to a perfectly matched probe. Tₘ is reduced by about 1° C. for each 1% of mismatching; thus, Tₘ, hybridisation, and/or wash conditions can be adjusted to hybridise to sequences of different degrees of complementarity. For example, sequences with ≧90% identity can be hybridised by decreasing the Tₘ by about 10° C. Generally, stringent conditions are selected to be about 5° C. lower than the Tₘ for the specific sequence and its complement at a defined ionic strength and pH. However, high stringency conditions can utilise a hybridisation and/or wash at, for example, 1, 2, 3, or 4° C. lower than the Tₘ; medium stringency conditions can utilise a hybridisation and/or wash at, for example, 6, 7, 8, 9, or 10° C. lower than the Tₘ; low stringency conditions can utilise a hybridisation and/or wash at, for example, 11, 12, 13, 14, 15, or 20° C. lower than the Tₘ. Using the equation, hybridisation and wash compositions, and desired Tₘ, those of ordinary skill will understand that variations in the stringency of hybridisation and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridisation of nucleic acids is found in Tijssen (Laboratory Techniques in Biochemistry and Molecular Biology-Hybridisation with Nucleic Acid Probes, Pt I, Chapter 2, Elsevier, New York, 1993), Ausubel et al., eds. (Current Protocols in Molecular Biology, Chapter 2, Greene Publishing and Wiley-Interscience, New York, 1995) and Sambrook et al. (Molecular Cloning: A Laboratory Manual, 2^{nd} ed., Cold Spring Harbor Laboratory Press, Plainview, N.Y., 1989).

The T6ODM nucleotide sequence variants identified by the present inventors encode a T6ODM enzymes with variant amino acid sequences. Accordingly in a further aspect, the present invention provides an isolated polypeptide comprising the amino acid sequence set forth in any one of SEQ ID NOs: 9 to 11.

In some embodiments, the present invention provides T6ODM amino acid sequence variants which display at least 50%, at least 55%, at least 60%, at least 65%, at least 70%, at least 75%, at least 80%, at least 85%, at least 90%, at least 91%, at least 92%, at least 93%, at least 94%, at least 95% at least 96%, at least 97%, at least 98%, at least 99% or 100% sequence identity to a polypeptide comprising an amino acid sequence set forth in any one of SEQ ID NOs: 9 to 11.

In some embodiments, the T6ODM amino acid sequence variants provided in accordance with this aspect of the invention do not consist of the amino acid sequence encoded by the reference sequence for T6ODM, as represented by GenBank accession number ADD85329.1.

When comparing amino acid sequences to calculate a percentage identity, the compared sequences should be compared over a comparison window of at least 50 amino acid residues, at least 100 amino acid residues, at least 150 amino acid residues, or over the full length of the relevant one of SEQ ID NOs: 9 to 11. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerised implementations of algorithms such as the BLAST family of programs as, for example, disclosed by Altschul et al. (Nucl. Acids Res. 25: 3389-3402, 1997). A detailed discussion of sequence analysis can be found in Unit 19.3 of Ausubel et al. (“Current Protocols in Molecular Biology” John Wiley & Sons Inc, 1994-1998, Chapter 15, 1998). Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

A used herein, the singular forms “a”, “an” and “the” include plural aspects unless the context already dictates otherwise.

“About” as used in the specification means approximately or nearly and in the context of a numerical value or range set forth herein means±10% of the numerical value or range recited or claimed.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of the steps or features.

Finally, reference is made to standard textbooks of molecular biology that contain methods for carrying out basic techniques encompassed by the present invention, including DNA restriction and ligation for the generation of the various genetic constructs described herein. See, for example, Sambrook and Russell, Molecular Cloning: A Laboratory Manual (3rd edition). Cold Spring Harbor Laboratory Press, 2001.

The present invention is further described by the following non-limiting examples. It is to be understood that the following description is for the purpose of describing particular embodiments only and is not intended to be limiting with respect to the above description.

### Example 1

### Production of Codeine, Oripavine and/or Morphine in P. bracteatum

The nucleotide sequence set forth in GenBank accession number GQ500139 (which encodes T6ODM) is cloned into a suitable expression vector, such as pBI121 (Clontech) under the control of the cauliflower mosaic virus 35S promoter (CaMV 35S) which directs constitutive expression in P. bracteatum. The transgene is introduced into P. bracteatum via Agrobacterium-mediated transformation using the method described in Solouki et al. (2009, supra). Transformants are selected using paromomycin and regenerated according to the method described in Solouki et al. (2009, supra). Transformants are then screened for alkaloid content.

In the transformants, strong constitutive expression of T6ODM, together with expression of COR is proposed to lead to the accumulation of codeine in the transformants.

In order to increase the accumulation of morphine in the transformants, CODM activity may also be increased in P. bracteatum in order to increase the conversion of codeine to morphine and also to increase the conversion of thebaine to orpiavine. Increasing the activity of CODM in the transformants may be achieved by transformation of P. bracteatum with the nucleotide sequence set forth in GQ500141 (which encodes CODM) using a similar method to that outlined above for transformation of P. bracteatum with T6ODM. In these embodiments, T6ODM and CODM may be contransformed into P. bracteatum on a single construct or, alternatively, the two transgenes may be sequentially transformed into P. bracteatum. Where sequential transformations are performed it may become necessary to introduce an additional selectable marker gene with each sequential transformation in order to allow selection of transformants transformed with both transgenes.

Conversely, in order to further reduce the level of morphine production in the transformants, and thus make plants with a higher yield or purity of codeine, any residual CODM activity in the plants may be reduced. Reduction of CODM activity in P. bracteatum may be achieved using suitable genetic modification of P. bracteatum. Suitable genetic modifications to reduce CODM expression may include, for example:

random mutagenesis such as transposon, chemical, UV and phage mutagenesis together with selection of mutants which underexpress CODM;

insertional mutagenesis of a CODM encoding nucleic acid with a knockout construct (for an example of targeted gene disruption in plants see Terada et al., Nat. Biotechnol. 20: 1030-1034, 2002);

post-transcriptional gene silencing (PTGS) or RNAi of a CODM encoding nucleic acid (for review of PTGS and RNAi see Sharp, Genes Dev. 15(5): 485-490, 2001; and Hannon, Nature 418: 244-51, 2002);

transformation of a cell with an antisense construct directed against a CODM encoding nucleic acid (for examples of antisense suppression in plants see van der Krol et al., Nature 333: 866-869; van der Krol et al., BioTechniques 6: 958-967; and van der Krol et al., Gen. Genet. 220: 204-212);

transformation of a cell with a co-suppression construct directed against a CODM encoding nucleic acid (for an example of co-suppression in plants see van der Krol et al., Plant Cell 2(4): 291-299);

transformation of a cell with a construct encoding a double stranded RNA directed against a CODM encoding nucleic acid (for an example of dsRNA mediated gene silencing see Waterhouse et al., Proc. Natl. Acad. Sci. USA 95: 13959-13964, 1998); and

transformation of a cell with a construct encoding an siRNA or hairpin RNA directed against a CODM encoding nucleic acid (for an example of siRNA or hairpin RNA mediated gene silencing in plants see Lu et al., Nucl. Acids Res. 32(21): e171; doi:10.1093/nar/gnh170, 2004).

As set out above, at least some cultivars of P. bracteatum express COR. However, where a cultivar does not express COR, or it is desired to increase the expression of COR in order to increase the yield of codeine, oripavine and/or morphine in transformed Papaver bracteatum, the expression of COR may be increased in the transformed Papaver bracteatum by transformation with a COR-encoding nucleotide sequence, as hereinbefore described. Again, the COR-encoding nucleotide sequence may be transformed into P. bracteatum using the method of Solouki et al. (2009, supra) and may be contransformed with T6ODM and/or CODM or may be sequentially transformed. Again, where sequential transformations are performed it may become necessary to introduce an additional selectable marker gene with each sequential transformation.

### Example 2

### Screening P. bracteatum for T6ODM, CODM and COR

A series of primers were utilised to amplify PCR products of different sizes at different locations in the T6ODM and CODM genes in P. somniferum. The same primers were also used to detect whether the T6ODM and CODM genes, or similar sequences, were present within the P. bracteatum genome. The PCR amplification conditions used were as follows: initial denaturation at 95° C. for 120 seconds; 30 cycles of denaturation at 95° C. for 30 seconds, primer annealing at 59° C. for 30 seconds and extension at 72° C. for 150 seconds; and a final elongation step of 72° C. for 5 minutes. Reactions were performed with the GoTaq Green Polymerase Mix (Promega) as per the manufacturer's instructions.

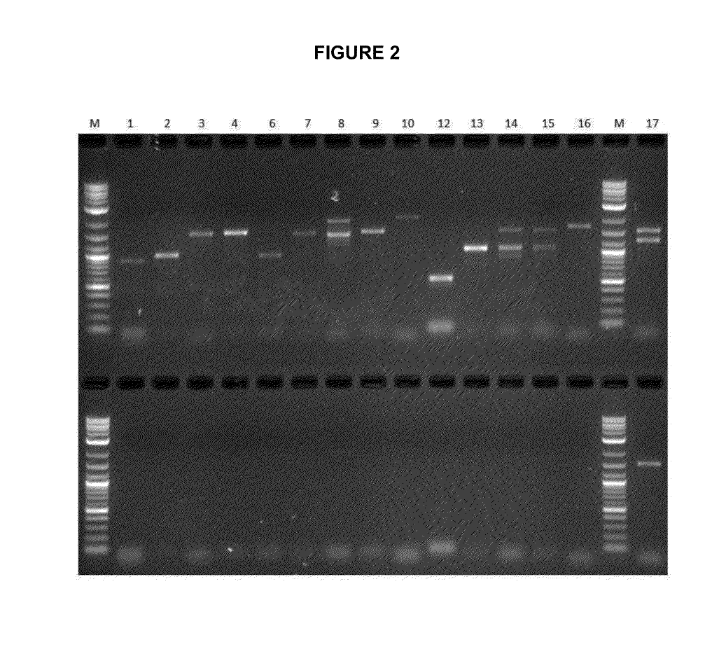

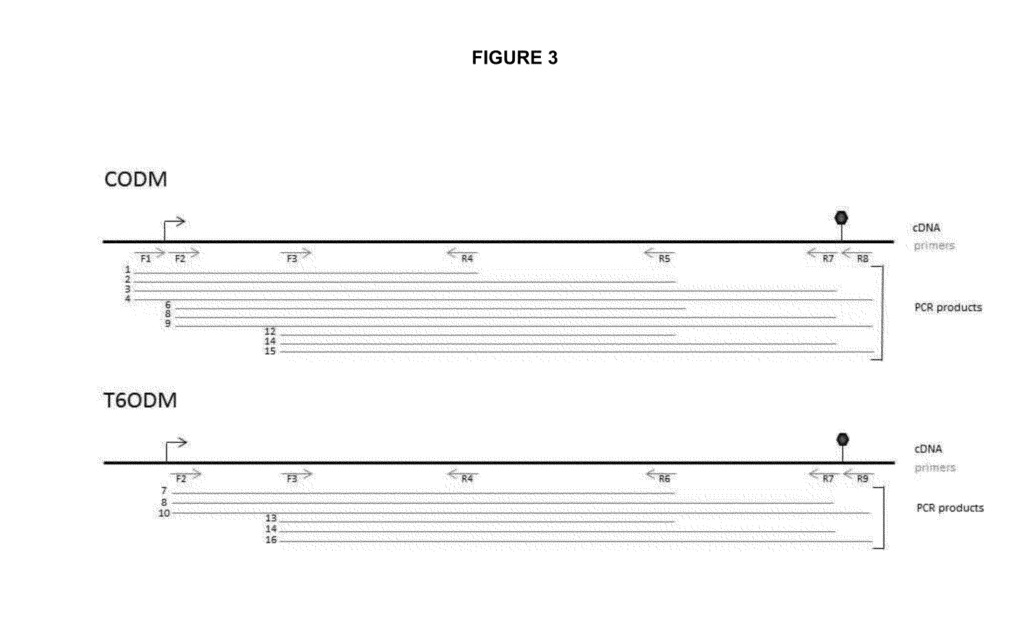

Table 2 below provides a summary of the primer combinations used to amplify various regions of the T6ODM and CODM genes. The results of the PCR amplifications are shown in FIG. 2 and the location of the primers in the CODM and T6ODM genes is shown in FIG. 3.

TABLE 2

| Gel Lane | Forward Primer | Reverse Primer |
|---|---|---|
| 1 | F1 (SEQ ID NO: 15) | R4 (SEQ ID NO: 16) |
| 2 | F1 (SEQ ID NO: 15) | R5 (SEQ ID NO: 17) |
| 3 | F1 (SEQ ID NO: 15) | R7 (SEQ ID NO: 18) |
| 4 | F1 (SEQ ID NO: 15) | R8 (SEQ ID NO: 19) |
| 6 | F2 (SEQ ID NO: 20) | R4 (SEQ ID NO: 16) |
| 7 | F2 (SEQ ID NO: 20) | R5 (SEQ ID NO: 17) |
| 8 | F2 (SEQ ID NO: 20) | R6 (SEQ ID NO: 21) |
| 9 | F2 (SEQ ID NO: 20) | R8 (SEQ ID NO: 19) |
| 10 | F2 (SEQ ID NO: 20) | R9 (SEQ ID NO: 22) |
| 12 | F3 (SEQ ID NO: 23) | R5 (SEQ ID NO: 17) |
| 13 | F3 (SEQ ID NO: 23) | R6 (SEQ ID NO: 21) |
| 14 | F3 (SEQ ID NO: 23) | R7 (SEQ ID NO: 18) |
| 15 | F3 (SEQ ID NO: 23) | R8 (SEQ ID NO: 19) |
| 16 | F3 (SEQ ID NO: 23) | R9 (SEQ ID NO: 22) |
| 17 | F10 (SEQ ID NO: 24) | R11 (SEQ ID NO: 25) |

As can be seen in FIG. 2, all PCR reactions were conducted using 200 ng of P. somniferum genomic DNA as template (top panel) gave rise to PCR products of the expected size.

However, no PCR products were obtained using 200 ng P. bracteatum genomic DNA as template (bottom panel). As a positive control, the COR1.1 gene was amplified from both genomes using the same methodology.

These results indicate that the P. bracteatum genome does not comprise a detectable level of the T6ODM and CODM genes, but does contain the COR1.1 gene.

### Example 3

### T6ODM and CODM Gene Identification and Isolation

The T6ODM and CODM genes were amplified from the genome of Papaver somniferum using the following primer sets.

(SEQ ID NO: 26)
5′-ATGGAGAAAGCAAAACTTATGAAGCTAGG-3′
and
(SEQ ID NO: 27)
5′-TTTCTGAAAGTAAAGGTACATTACATGTGG-3′
for T6ODM;
and
(SEQ ID NO: 28)
5′-ATCTGACAAGAAAGTTCATCAAATATAGAGTTC-3′;
and
(SEQ ID NO: 29)
5′-CTTATTTCATCATATATTAAACACAATGTGGAG-3′
for CODM.

Genomic DNA was isolated from Papaver somniferum by grinding tissue samples in liquid nitrogen and mixing with three times the sample volume of extraction buffer (1% sarcosyl, 100 mM Tris-Cl, 100 mM NaCl, 10 mM EDTA, pH 8.5) followed by phenol chloroform extraction, precipitation with 0.1 volumes 3M sodium acetate (pH 4.8) and 1 volume 100% isopropanol, washing with 1 mL 70% ethanol and resuspension in an appropriate volume of sterile distilled water with 40 μg/mL RNAse A.

The PCR conditions were as follows: initial denaturation at 98° C. for 30 seconds; 25 cycles of denaturation at 98° C. for 10 seconds, primer annealing at 59° C. for 15 seconds and extension at 72° C. for 120 seconds; and a final elongation step of 72° C. for 10 minutes. Reactions were performed with the Phusion High-Fidelity DNA Polymerase (Thermo Scientific) as per the manufacturer's instructions using 200 ng of genomic DNA as template. PCR products were gel extracted (Qiagen), cloned into pGEM-T Easy (Promega) and transformed into JM109 competent cells (Promega), all according to the manufacturer's instructions. The cloned products were sequenced and this revealed a 2288 bp T6ODM gene (relative to the 1095 bp T6ODM mRNA) and a 1663 bp CODM gene (relative to the 1083 bp CODM mRNA).

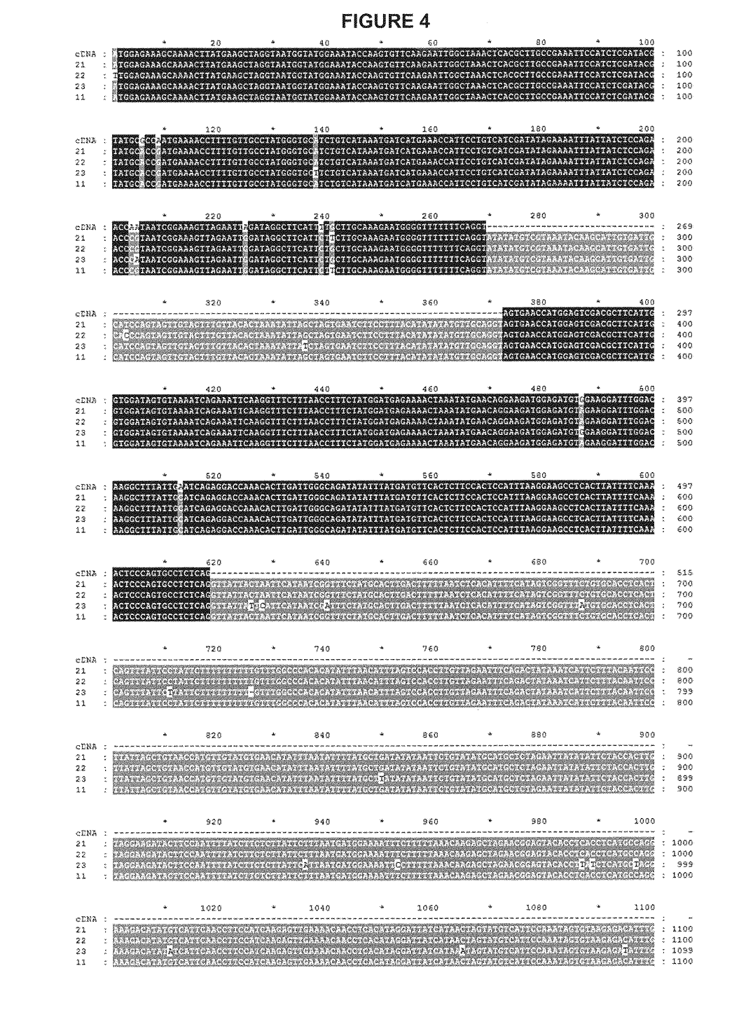

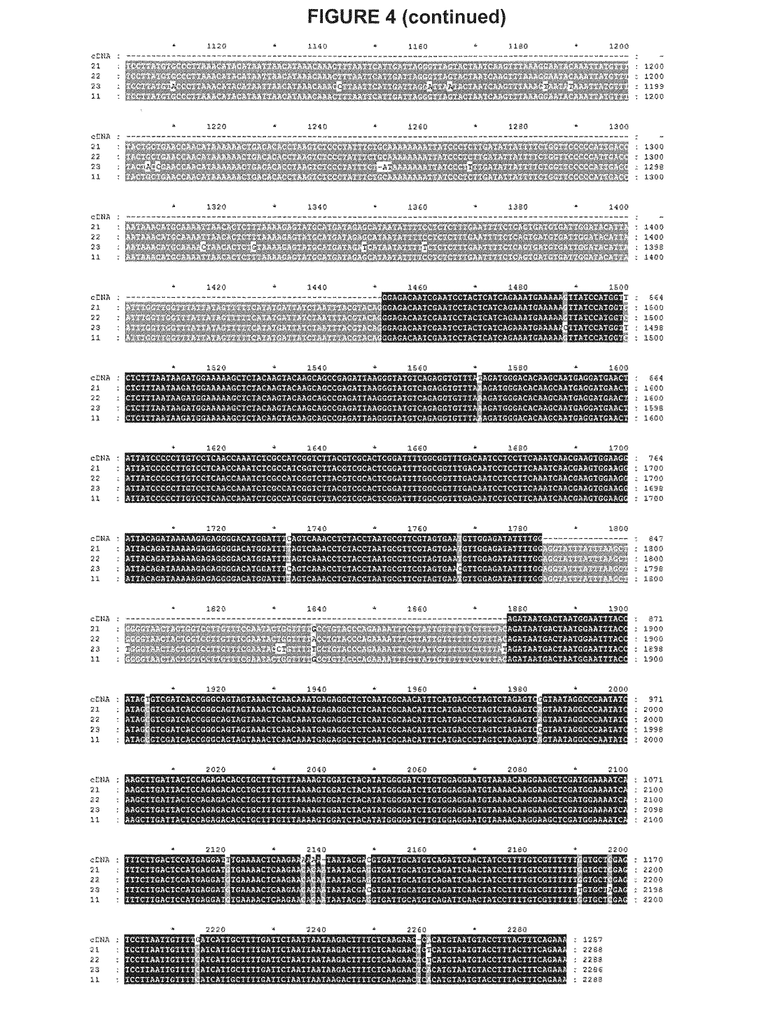

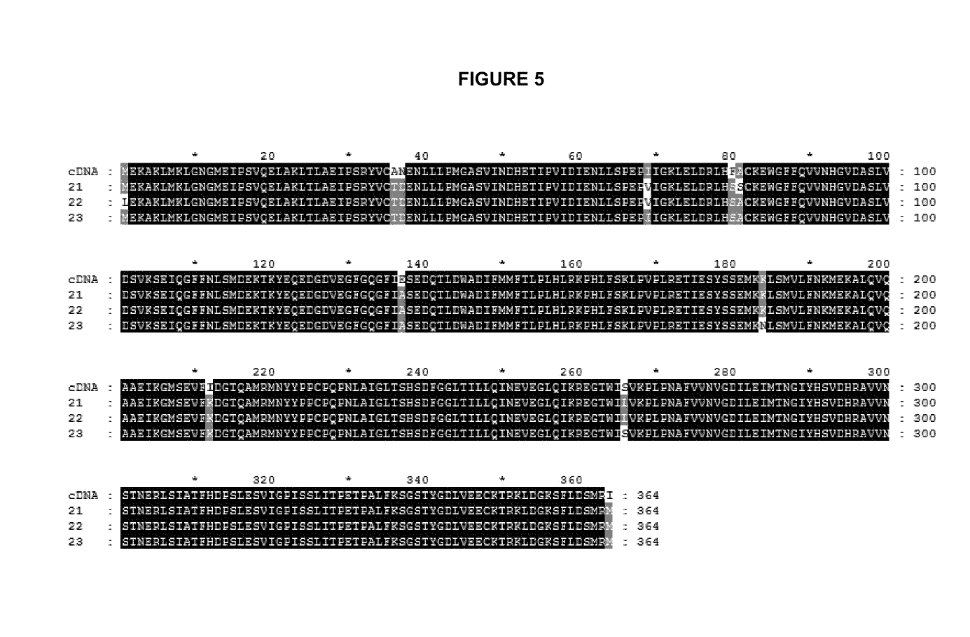

Sequencing of a number of cloned products led to the identification of sequence variants when compared to Genbank reference cDNA sequences for the T6ODM (GQ500139) and CODM (GQ500141) genes. For example, four nucleotide sequence variants of the T6ODM gene were identified which respectively comprise the cDNA nucleotide sequences set forth in SEQ ID NOs: 1 to 4. These cDNA nucleotide sequence variants were derived from genomic DNA clones comprising the nucleotide sequences set forth in SEQ ID NOs: 5 to 8, respectively. These sequence variants collectively encode T6ODM enzyme variants comprising the amino acid sequences set forth in SEQ ID NOs: 9 to 11. FIGS. 4 and 5 show nucleotide and amino acid sequence alignments, respectively, of the T6ODM variants identified in comparison to the reference sequence for T6ODM (i.e. GQ500139 and the encoded polypeptide represented by GenBank accession number ADD85329.1).

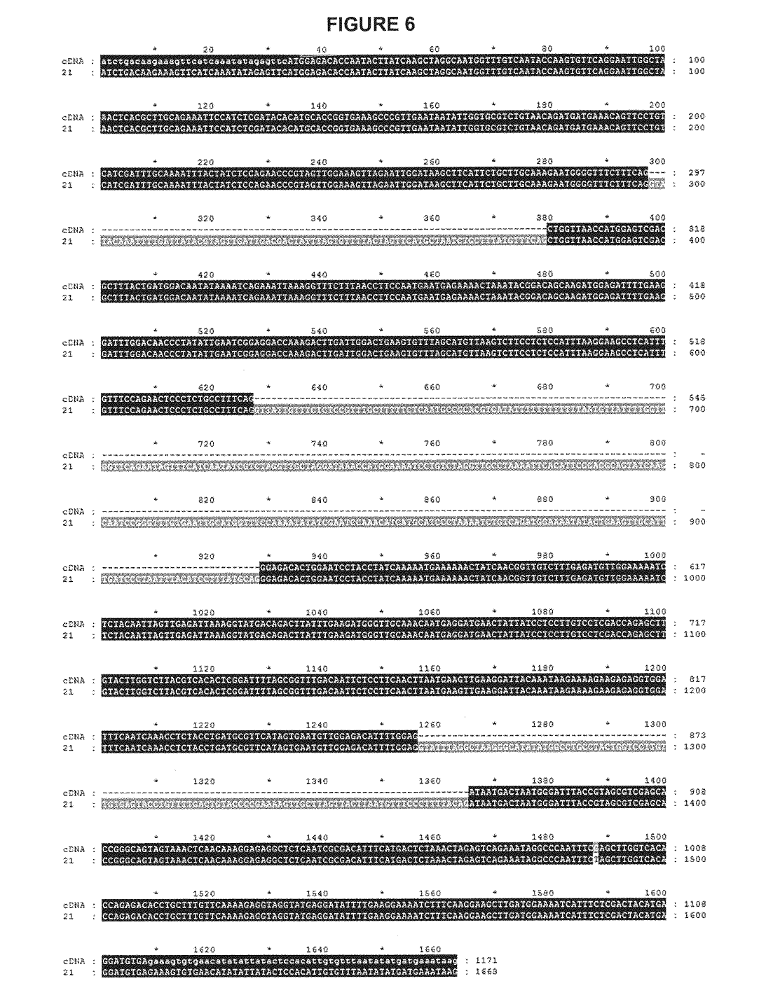

A single nucleotide sequence variant of the CODM gene was identified which comprises the cDNA nucleotide sequence set forth in SEQ ID NO: 12. This cDNA nucleotide sequence variant was derived from a genomic DNA clone comprising the nucleotide sequences set forth in SEQ ID NOs: 13. However, this nucleotide sequence variant encodes a CODM enzyme with the same amino acid sequence as that encoded by the CODM reference, as represented by GenBank accession number ADD85331.1. FIG. 6 shows a nucleotide sequence alignment of the CODM variant identified in comparison to the reference sequence for CODM (i.e. GQ500141).

### Example 4

### Construct Assembly

The coding sequences of T6ODM, CODM and codeinone reductase 1.1 (COR1.1) were synthesised commercially (Mr Gene: www.mrgene.com) based on the previously published cDNA sequences from Hagel and Facchini (Nat. Chem. Biol. 6: 273-275, 2010) and Unterlinner et al. (Plant J. 18: 465-475, 1999). In the synthesised constructs, the cDNA sequences were placed upstream of the terminator region of the nopaline synthase (nos) gene, which contains a signal for the termination of gene transcription (Bevan et al., Nucl. Acids Res. 11: 369-385, 1983). The cauliflower mosaic virus 35S promoter with dual enhancer motifs was amplified from the pMDC32 plasmid constructed by Curtis and Grossniklaus (Plant Phys. 133: 462-469, 2003) and inserted upstream of the cDNA sequences to drive expression. This 35S promoter was obtained by PCR using the 5′-CAAACGCGTCAGGTCAACATGGTGGAGCAC-3′ (SEQ ID NO: 30) and 5′-CAAAGATCTGATCCTCTAGAGTCGAGGTCCTCTC-3′ (SEQ ID NO: 31) primers, which contain the MluI and BglII restriction enzyme recognition sites, respectively, to amplify the promoter from 5 ng of the pMDC32 plasmid. The PCR amplification conditions used were as follows: initial denaturation at 98° C. for 30 seconds; 30 cycles of denaturation at 98° C. for 10 seconds, primer annealing at 72° C. for 15 seconds and extension at 72° C. for 60 seconds; and a final elongation step of 72° C. for 10 minutes. Reactions were performed with the Phusion High-Fidelity DNA Polymerase (Thermo Scientific) as per the manufacturer's instructions. PCR products were gel extracted (Qiagen), cloned into pGEM-T Easy (Promega) and transformed into JM109 competent cells (Promega), all according to the manufacturer's instructions. The sequence of the amplified promoter is set forth in SEQ ID NO: 14. The promoter was excised from pGEM-T Easy by restriction digest and inserted upstream of the cDNA sequences by ligating the fragment into the MluI and BglII restriction enzyme recognition sites in the synthetic vectors from Mr Gene.

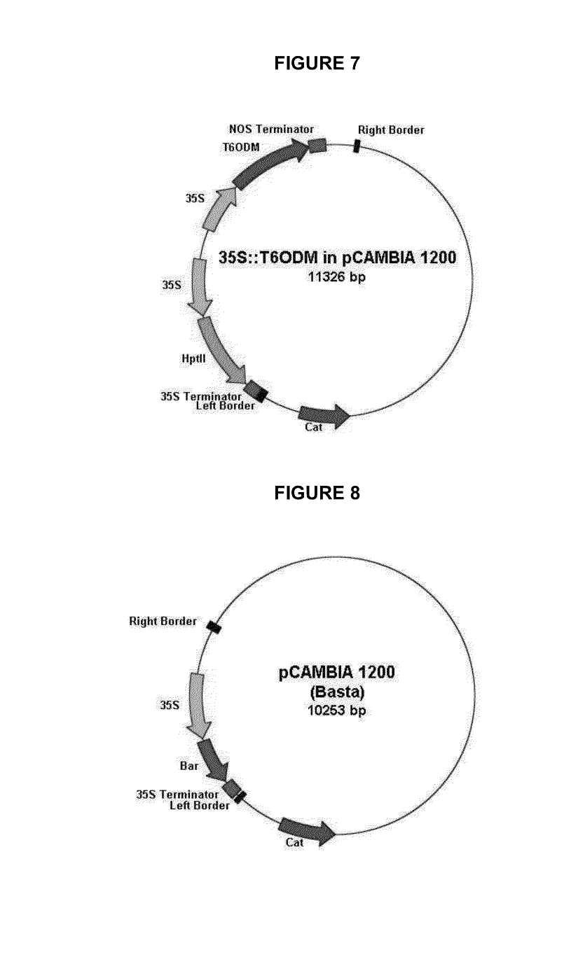

The 35S::cDNA::nos terminator DNA cassette was excised by restriction enzyme digestion and inserted into the multiple cloning site of the pCAMBIA 1200 plant transformation vector. The 35S::T6ODM::nos terminator DNA cassette was inserted into the native pCAMBIA 1200 vector containing the selectable marker gene hptII that encodes hygromycin phosphotransferase. A vector map for the T6ODM construct is shown in FIG. 7.

The 35S::CODM::nos terminator and the 35S::COR1.1::nos terminator DNA cassettes were transferred into modified pCAMBIA 1200 vectors that had been modified to confer resistance to glufosinate and bispyribac sodium (BPS). The Basta-resistance gene (pat) was isolated by PCR from the pTOOL2 vector (Invitrogen) and a BPS-resistance gene allele (acetolactate synthase—als), originally described by Oldach et al. (Annals of Botany 101: 997-1005, 2008), was isolated from seeds of a BPS-resistant Medicago variety (“Angel”).

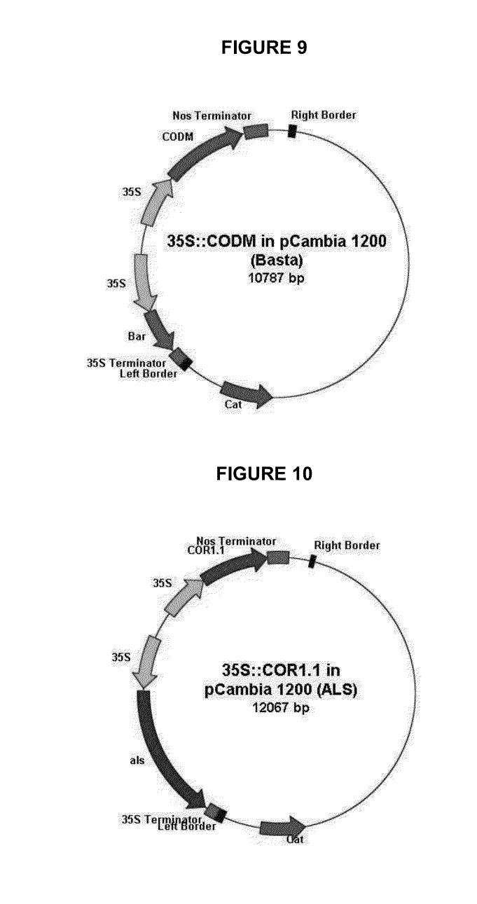

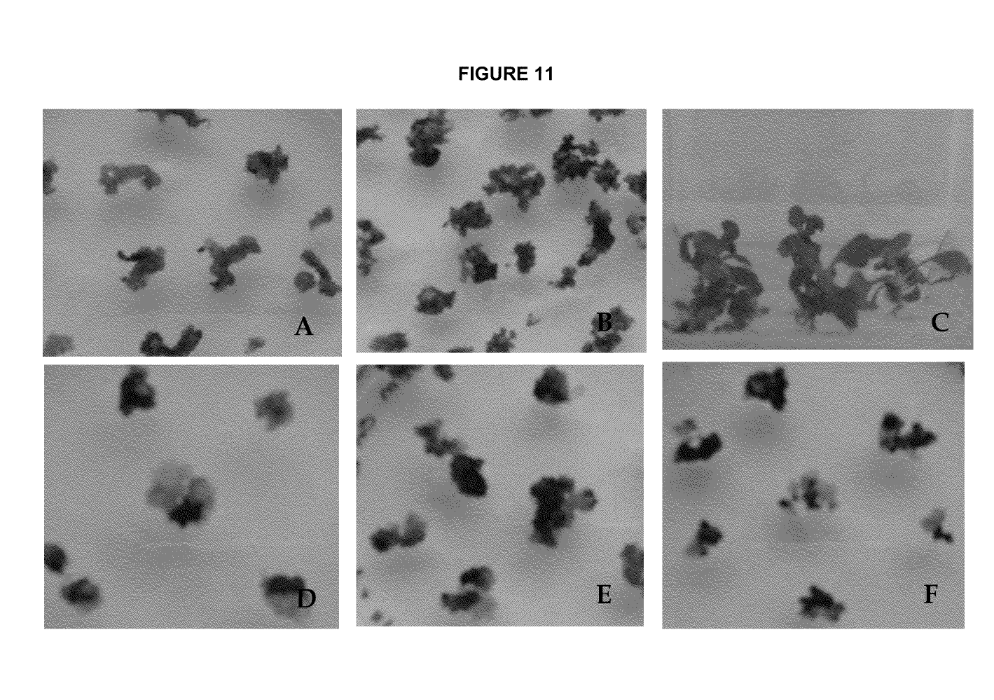

Both resistance genes were isolated by PCR with the Phusion High-Fidelity DNA Polymerase (Thermo Scientific) as per the manufacturer's instructions. PCR products were gel extracted (Qiagen), cloned into pGEM-T Easy (Promega) and transformed into JM109 competent cells (Promega), all according to the manufacturer's instructions. The genes were excised from pGEM-T Easy by restriction digest with XhoI and inserted into the pCAMBIA 1200 vector backbone, from which the hygromycin resistance gene had been previously excised by digestion with XhoI. The pat gene was amplified from 5 ng of pTOOL2 DNA with the 5′-GATCTCGAGCTTTCGATGAGCCCAGAACGACGC-3′ (SEQ ID NO: 32) and 5′-AAACTCGAGCTTGTCGATCGACATCAGATTTCGGTGACGGG-3′(SEQ ID NO: 33) primers and the following amplification conditions: initial denaturation at 98° C. for 30 seconds; 25 cycles of denaturation at 98° C. for 10 seconds, primer annealing at 60° C. for 15 seconds and extension at 72° C. for 60 seconds; and a final elongation step of 72° C. for 10 minutes. The als gene was amplified from 100 ng of “Angle” genomic DNA with the 5′-GTTCTCGAGCTTTCGATGGCAGCCACCACCACC-3′(SEQ ID NO: 34) and 5′-TTACTCGAGCTTGTCGATCGACATCAATAACTCCTTCTTCCATCACC-3′ (SEQ ID NO: 35) primers and the following amplification conditions: initial denaturation at 98° C. for 30 seconds; 25 cycles of denaturation at 98° C. for 10 seconds, primer annealing at 62° C. for 15 seconds and extension at 72° C. for 120 seconds; and a final elongation step of 72° C. for 10 minutes. Vector maps showing the bar, CODM and COR1.1 constructs are shown in FIGS. 8 to 10, respectively.

### Example 5

### Seed Sterilisation and Germination

Seeds of Papaver somniferum and two varieties of P. bracteatum (P. bracteatum var. PB-1 and P. bracteatum var. PB-2; see WO 2009/092134) were washed by stirring in 70% ethanol for 5 mins, followed by 3 rinses in sterile Milli-Q water. The seeds were then stirred in 5% (v/v) sodium hypochlorite solution supplemented with few drops of Tween 20 for 7 mins, followed by 5 rinses with sterile Milli-Q water. Surface sterilised seeds were planted on ½ MS (Murashige and Skoog) medium with Gamborg's vitamins, 3% sucrose and 0.3% phytagel. The pH was adjusted to 5.7 with 1 M KOH. The culture plates were incubated at 22° C. D/N under 10-h light/14-h dark cycle for seed germination.

### Example 6

### Callus Induction

One week old seedlings were transferred to the callus induction medium (CIM) consisting of ¾ MS, 3% sucrose, casein (300 mgL⁻¹), myoinositol (100 mg L⁻¹) and 0.3% phytagel. The pH was adjusted to 5.7 and the medium was sterilized by autoclaving at 121° C. for 20 minutes. After autoclaving the medium was supplemented with filter sterilised (Millex®GP Filter Unit 0.22 μM) stock solutions of 2,4-dichlorophenoxy acetic acid (1 mg L⁻¹), kinetin (0.1 mg L⁻¹) and ascorbic acid (15 mg L⁻¹). The plates were kept in dark at 20° C. The calli were subcultured every two weeks on fresh medium.

### Example 7

### Agrobacterium Transformation

Agrobacterium tumefaciens strain AGL0 was used for transformations. Bacterial cells were maintained in 40% glycerol stock at −80° C. Freeze-thaw method was used for transforming AGL0 competent cells with various binary vectors (Table 3).

TABLE 3
Binary constructs used for
transforming A. tumefaciens strain AGL0

| | | | Selection | |
|---|---|---|---|---|
| Vector | Backbone | Gene | Bacteria | Plant |
| 1 | pCambia1200 | 35S::BAR | chloramphenicol | Basta |
| 2 | pCambia1200 | 35S::T6ODM | chloramphenicol | Hygromycin |
| 3 | pCambia1200 | 35S::CODM | chloramphenicol | Basta |
| 4 | pCambia1200 | 35S::COR1.1 | chloramphenicol | Bis-pyribac |
| | | | | sodium |

Tubes of frozen AGL0 competent cells were allowed to thaw at room temperature. 5 μl of the plasmid DNA was added to 100 μl of AGL0 and mixed by gently flicking the tubes. The bacterial cells were frozen in liquid nitrogen for 1 min and then subjected to heat shock in a water bath at 37° C. for 5 mins. 500 μl of LB (Luria-Bertani) broth was added and the tubes were incubated for 2 hrs at 28° C. with shaking (150 rpm). 150 μl of each transformation culture was plated on to duplicate LB agar medium supplemented with antibiotics, Rifampicin (50 μg ml⁻¹) and Chloramphenicol (100 μg ml⁻¹). The plates were incubated at 28° C. for 2-4 d. Colony PCR was carried out for amplifying genes of interest and positive colonies were selected for plant transformation.

### Example 8

### Callus Transformation

PCR selected agrobacterium colonies were inoculated in 10 ml LB broth supplemented with antibiotics, Rifampicin (50 μg ml⁻¹) and Chloramphenicol (100 μg ml⁻¹) and incubated at 28° C. for 4 d. The bacterial culture was centrifuged at 4° C., 4000 rpm for 10 mins and cells were harvested. Agrobacterium infection solution was prepared by adding small volumes of harvested cells to filter sterilized (Millipore Express™PLUS 0.22 μM) pre-treatment medium (MS 4.4 g L⁻¹, glucose 18 g L⁻¹, sucrose 30 g L⁻¹, acetosyringone 10 μM, pH 5.2) and the OD₆₀₀ was adjusted between 0.5 to 1.5.

6-8 week old calli were used for transformation (see FIG. 11A). The calli were incubated in pre-treatment medium for 5 mins, and then transferred to agrobacterium infection solution for 5 mins. The infected calli were removed from infection solution and placed on sterile filter paper for drying inside the laminar flow-hood. Once dry, the calli were transferred to co-cultivation medium (MS 4.4 g L⁻¹, glucose 10 g L⁻¹, sucrose 30 g L⁻¹, casein 300 mg L⁻¹, myoinositol 100 mg L⁻¹ acetosyringone 10 μM, 2,4-D 1 mg L⁻¹, kinetin 0.1 mg L⁻¹, ascorbic acid 15 mg L⁻¹, pH 5.2). The culture plates with transformed calli were incubated in dark in the Sanyo versatile environmental chamber (MLR-351) at 20° C. D/N for 3 d.

### Example 9

### Selection of Transformants and Regeneration

After 3 days of co-cultivation with agrobacterium, the calli were washed 5 times with pre-treatment medium without acetosyringone and with the antibiotic cefotaxime (400 mg L⁻¹) for arresting agrobacterium growth. The washed calli were blotted dry on sterile filter paper and transferred to regeneration medium The regeneration medium consists of ¾ MS, sucrose 30 g L⁻¹, Phytagel 0.3%, pH 5.7, after autoclaving this medium was supplemented with filter sterilised stock solutions of naphthalene acetic-acid-1 mg L⁻¹, benzylaminepurine-0.5 mg L⁻¹, cefotaxime-400 mg L⁻¹ and appropriate concentration of the selection agent (Table 4). The calli were maintained under selection (see FIGS. 11 D, E and F) and subcultured every two weeks on fresh medium until shoot regeneration was observed.

TABLE 4
The concentrations of selection agents determined by performing
kill curve experiments on all three varieties of Papaver spp.

| Constructs | Selection agent | Concentration |
|---|---|---|
| 35S::BAR | Basta | 10 mg L⁻¹ |
| 35S::CODM | Basta | 10 mg L⁻¹ |
| 35S::T6ODM | Hygromycin | 50 μg ml⁻¹ |
| 35S::COR1.1 | Bis-pyribac sodium | 80 μg L⁻¹ |

After shoot regeneration (FIG. 11B) and formation of cotyledons, the regenerants were transferred to rooting medium prepared in culture vessel MAGENTA® with vented lids (FIG. 110). The rooting medium contains ½ MS, 3% sucrose, 0.3% phytagel. pH was adjusted to 5.7 and appropriate selection was added to the medium after autoclaving.

As indicated above, FIG. 11 provides photos evidencing callus induction and plant regeneration of P. bracteatum var PB-1.

### Example 10

### Screening of Transformants

Regenerated P. bracteatum plantlets can be screened for the presence and/or expression of the transgene using standard techniques known in the art (e.g. PCR, Q-PCR, Southern blot, Northern blot). However, this is not entirely necessary given that identification of the presence of codeine, oripavine and/or morphine in the transgenic plants will in effect represent confirmation that the transgene is present and is being expressed.

In this regard, transformed P. bracteatum plants were grown for approximately three months, together with wild-type “control” P. bracteatum plants. Five small immature plants (approx. 0.10 g in total) from each group were collected and combined, and the combined plant material was macerated using a mortar and pestle. The alkaloid was extracted from the combined plant in 5 ml of 10% acetic acid solution. In this regard, the liquid was separated from the plant matter, with the extracted liquid sample being used for the subsequent alkaloid content analysis.

The extracted liquid samples were screened for alkaloid content using an Agilent 1200 HPLC system using absorbance measured at 280 nm. The system used gradient elution according to the manner shown in Table 5 below. The HPLC column used was a Phenonex Luna Column with the following specifications:
a. C18, 5 μm, Encapped
b. id: 4.6 mm
c. Length=150 mm

TABLE 5
Gradient elution HPLC conditions

| Time | % Eluent A | % Eluent B |
|---|---|---|
| 0 | 90 | 10 |
| 10 | 60 | 40 |
| 11 | 20 | 80 |
| 16 | 20 | 80 |
| 17 | 90 | 10 |
| 26 | 90 | 10 |

Note:
Eluent A: 0.1% TFA in Water; Eluent B: 0.1% TFA in Acetonitrile; Flow Rate = 1.0 mL/min

100 μl of the extracted liquid sample was injected into the HPLC column. Calibration Standards of morphine, codeine, oripavine and thebaine were run prior to the plant samples to determine compound identification. Each sample was screened for the four alkaloids. The results of this analysis are shown in Table 6.

TABLE 6
Alkaloid content analysis of transformed plants

| Plant | Morphine | Codeine | Oripavine | Thebaine |
|---|---|---|---|---|
| Control | x | x | x | ✓ |
| T6ODM | x | ✓ | x | ✓ |
| CODM | x | x | ✓ | ✓ |
| CODM + T6ODM | ✓ | ✓ | ✓ | ✓ |

Note:
x Alkaloid absent; ✓Alkaloid present

As can be seen from the table above, control plants (i.e. plants lacking the T6ODM and CODM genes) fail to produce the alkaloids morphine, codeine and oripavine. The control plants do however produce thebaine as expected. Plants transformed with the T6ODM construct were shown to produce the alkaloid codeine, thereby establishing that the pathway from thebaine to codeine had been restored in this transformant. Plants transformed with the CODM construct were shown to produce oripavine, thereby establishing that the pathway from thebaine to oripavine had been restored in this transformant. Finally, plants transformed with the CODM and T6ODM constructs were able to produce all four alkaloids confirming that the pathway from thebaine to morphine (see FIG. 1) had been restored.

The above alkaloid analysis procedure can also be performed on more mature plants with root tissue, leaf tissue, latex and/or capsules being assayed for alkaloid content.

We claim:

1. A genetically modified poppy plant of the species Papaver bracteatum, or a hybrid thereof, wherein the expression of one or more of thebaine 6-O-demethylase, codeine O-demethylase and/or codeinone reductase is increased relative to wild type P. bracteatum and wherein said genetically modified poppy plant produces an increased quantity of an alkaloid selected from codeine, oripavine and/or morphine relative to a wild type P. bracteatum.

2. The poppy plant of claim 1, wherein at least the expression of thebaine 6-O-demethylase is increased relative to wild type P. bracteatum.

3. The poppy plant of claim 1, wherein the poppy plant comprises one or more transgenes which encode thebaine 6-O-demethylase, codeine O-demethylase and/or codeinone reductase.

4. The poppy plant of claim 3, wherein the one or more transgenes comprise the nucleotide sequence set forth in any one or more of SEQ ID NOs: 1 to 8, 12 and 13.

5. The poppy plant of claim 1, wherein the thebaine 6-O-demethylase comprises the amino acid sequence set forth in one of SEQ ID NOs: 9 to 11.

6. The poppy plant of claim 1, wherein the plant produces seed capsules and an alkaloid selected from codeine, oripavine and/or morphine in the first growing season after germination in a temperate poppy growing region.

7. The poppy plant of claim 1, wherein the plant produces seed capsules and an alkaloid selected from codeine, oripavine and/or morphine in the first growing season after germination at a latitude of between 40° and 44°.

8. The poppy plant of claim 1, wherein the plant comprises an indehiscent seed capsule or a dehiscent seed capsule.

9. A progeny plant having a poppy plant of claim 1 as a parent, or a mutant or derivative plant of the poppy plant of claim 1, wherein the progeny plant or mutant or derivative plant comprises increased expression of one or more of thebaine 6-O-demethylase, codeine O-demethylase and/or codeinone reductase relative to wild type P. bracteatum and wherein said mutant or derivative produces an increased quantity of an alkaloid selected from codeine, oripavine and/or morphine relative to a wild type P. bracteatum.

10. Reproductive material, latex or an isolated cell derived from, or straw or straw concentrate produced from, the plant of claim 1.

11. The reproductive material of claim 10, wherein the reproductive material comprises a seed.

12. An in-vitro culture comprising one or more of the isolated cells of claim 10.

13. The in-vitro culture of claim 12, wherein said culture produces an alkaloid selected from codeine, oripavine and/or morphine.

14. A method of producing an alkaloid selected from codeine, oripavine and/or morphine, the method comprising growing a plant according to claim 1 such that the plant produces an alkaloid selected from codeine, oripavine and/or morphine; and extracting the codeine, oripavine and/or morphine from the poppy plant or a part thereof.

15. An alkaloid selected from codeine, oripavine and/or morphine produced according to the method of claim 14.

16. An expression construct comprising one or more transgenes which encode thebaine 6-O-demethylase, codeine O-demethylase and/or codeinone reductase, wherein said one or more transgenes are operably connected to a transcriptional control sequence which is active in P. bracteatum.

17. The expression construct of claim 16, wherein the one or more transgenes comprise the nucleotide sequence set forth in any one or more of SEQ ID NOs: 1 to 8, 12 and 13.

18. The expression construct of claim 16, wherein the thebaine 6-O-demethylase comprises the amino acid sequence set forth in one of SEQ ID NOs: 9 to 11.

19. An isolated nucleic acid molecule comprising:
(i) the nucleotide sequence set forth in any one of SEQ ID NOs: 1 to 8, 12 and 13;
(ii) a nucleotide sequence which is at least 50% identical to the nucleotide sequence set forth in any one of SEQ ID NOs: 1 to 8, 12 and 13; or
(iii) a nucleotide sequence which hybridizes to a nucleic acid molecule comprising the nucleotide sequence set forth in any one of SEQ ID NOs: 1 to 8, 12 and 13 under stringent conditions.

20. An isolated polypeptide comprising:
(i) the amino acid sequence set forth in one of SEQ ID NOs: 9 to 11; or
(ii) an amino acid sequence which is at least 50% identical to the amino acid sequence set forth in any one of SEQ ID NOs: 9 to 11.
